(19) United States
(12) Patent Application Publication
(10) Pub. No.: US 2007/0105102 A1
(43) Pub. Date: May 10, 2007

(54) Method for detection and characterization of pre-malignant transformation

(75) Inventors: Sancy Leachman, Park City, UT (US); Christos Hatzis, Cambridge, MA (US); Nandan Padukone, Melrose, MA (US); Harold Erickson, Salt Lake City, UT (US); Patricia Porter-Gill, Laytonsville, MD (US)

Correspondence Address: HELLER EHRMAN LLP, 1717 RHODE ISLAND AVE, NW, WASHINGTON, DC, 20036-3001, US

(73) Assignees: University of Utah Reasearch Foundation, Salt Lake City, UT (US); Nuvera Biosciences, Inc., Woburn, MA (US)

(21) Appl. No.: 10/554,782

(22) PCT Filed: Apr. 29, 2004
(86) PCT No.: PCT/US04/13318

§ 371 Date: Nov. 21, 2006

Related U.S. Application Data
(60) Provisional application No. 60/466,061, filed on Apr. 29, 2003.

(51) Int. Cl. C12Q 1/68; G01N 33/574; A61K 48/00

(52) U.S. Cl. 435/6; 435/7.23; 514/44

(57) ABSTRACT

Methods are provided for estimating the risk of developing melanoma and related malignancies in an individual. Methods and compositions for diagnosing, treating, and preventing melanoma and related malignancies also are provided.

This application claims priority to U.S. application Ser. No. 60/466,061, filed Apr. 29, 2003, the contents of which are hereby incorporated by reference in their entirety.

### FIELD OF THE INVENTION

The present invention relates to a pathway of genes and proteins and their role in pre-malignant transformation. More specifically, the invention relates to differential expression of genes related mainly to an oxidative stress pathway that are involved in melanoma. The invention provides methods and compositions that are useful in diagnosing, treating, and/or preventing cancer, including melanoma.

### BACKGROUND OF THE INVENTION

Melanoma is a devastating malignancy with one of the most rapidly increasing mortality rates of any cancer. Melanoma is the most lethal skin cancer globally, affects over 50,000 Americans with an estimated death of 9000 per year. Familial melanoma that refers to the clustering of several cases within a single family accounts for only 6-12% of melanoma; however, a family history of melanoma is associated with a 30-70-fold increase in relative risk. In contrast to other malignancies, melanoma often affects patients in their third and fourth decades of life. Chemotherapy is rarely successful and five-year survival rates for patients with metastatic melanoma are only 5-10%.

Many families exhibiting an increased incidence of melanoma have a mutation in the p16 gene, a cell cycle inhibitor previously identified as a melanoma pre-disposition gene. In general, three genes have been shown to have co-segregating germline mutations in familial melanoma kindreds: p16, CDK4 (only three families), and ARF (only two families). CDK4 and ARF were discovered by candidate gene approaches based on the prior identification of p16 by linkage analysis. Studies of families in the Utah Population Database have previously described the association of germline p16 mutations with familial melanoma and have suggested that 10-50% of families with increased incidence of melanoma have mutations in this gene. In addition, p16 mutations are found in about 10-25% of sporadic primary melanomas, suggesting that disruption of the p16/Rb pathway is a common early event in the development of melanoma.

An understanding of the molecular genetic basis of melanoma permits development of specifically targeted diagnostic and therapeutic approaches. Therapies that target specific molecular pathways have come into the limelight only recently. The ability to better understand relationships of molecular mechanisms in complex disease processes continues to be a focus of attention in light of the promising advances made overall by targeted therapies. Elucidating disease mechanisms has involved deciphering roles of individual genes or proteins in complex inter-dependent pathways. Not only does such work require an initial hypothesis regarding a potential gene/protein but also considerable laboratory experimentation in detailing the biological system. The availability of a methodology to “reverse-engineer” pathways from molecular profiles in disease would help this significantly. A library of tumor and normal tissue samples could be used to measure gene expression and protein interactions/modifications and the resulting information can be systematically integrated into pathways that link the differentially regulated genes and proteins through their interactions to the specific response.

At present, the ability to accurately and reproducibly identify suspicious nevi is very limited. The present method of choice uses surface microscopy and the analysis of digital images. These systems are very costly and have not been proven to be exceptionally effective. They are also very labor intensive and require a great deal of expertise for interpretation. The ability to detect a dangerous mole with an accurate, non-invasive technique would revolutionize the field. For example, such non-invasive methods may allow anyone, a family practitioner or perhaps even the patient themselves, to identify lesions that need to be removed. Accordingly, such methods are greatly to be desired.

Of all measures of the primary lesion, the best prognostic indicators presently available are the thickness of the lesion and whether the lesion is ulcerated. Methods for evaluating the expression of a panel of genes would provide a better estimate of metastatic potential and therefore allow better prognostic methods. Accordingly, such methods would be highly desirable.

In addition, methods that profile the expression of multiple genes in patient tissue would also permit evaluation of chemoprevention and anti-cancer therapies. In particular, such methods would offer improved sensitivity and specificity over current non-specific laboratory tests or screening with specific tumor antigens or laboratory tests such as PSA or LDH levels. Moreover, multidrug regimens are commonly utilized in the treatment of cancer, targeting multiple biochemical pathways in cancer cells. Methods of determining the expression of a panel of genes would provide guidance as to the choice of drugs that could be most effectively used in a multi-drug regimen.

It is apparent, therefore that methods that identify the gene expression profile in patient tissue that is cancerous or that is suspected to be cancerous would be highly advantageous. Methods for modifying the gene expression profile also would be advantageous.

### SUMMARY OF THE INVENTION

It is therefore an object of this invention to provide methods for predicting the risk of the development of tumors, such as melanoma.

It is a further object of this invention to provide methods of treating tumors such as melanoma and of reducing tumor recurrence.

In accomplishing these objects there is provided a method of detecting a tumor, such as melanoma, or a pre-malignant transformation in a mammal, comprising assaying the level of expression of at least one of a set of target genes, and in particular oxidative stress pathway genes, in a sample obtained from the mammal. The presence of melanoma or a pre-malignant transformation also can be indicated by the altered expression of any of the target genes in the sample or by measuring changes in the activity of the protein product of any of the genes. The genes can be selected from the gene panel consisting of the target genes listed in Tables 1, 2 and/or 5.

In one aspect of the invention, the altered expression of any of the target genes in a sample is determined by a method selected from the group consisting of: genetic microarray analysis; quantitative PCR; assay of the level of protein expression in a sample including Western blot, ELISA; mRNA detection methods including RT-PCR, Northern hybridization; post-translational protein modification; 2-D electrophoresis for kinase, phosphorylation, glycosylation, and prenylation assays; and other biochemical assays designed to detect specific enzymatic activities of selected members of the gene panel.

In another aspect, the invention provides methods to determine the level of protein expression in a sample, for example, a skin tissue or a bodily fluid, wherein the proteins are soluble proteins, wherein the level of protein expression is determined via a binding assay including ELISA.

In another aspect, the invention provides methods of inhibiting or preventing tumorigenesis or growth of a tumor such as melanoma, comprising administering to a patient suffering from melanoma a composition that modifies expression of a target gene listed in Tables 1, 2 and/or 5, wherein the composition modifies the expression of a target gene that induces tumorigenesis or growth of a tumor such as melanoma, for example by inhibiting expression of the target gene.

In another aspect, the composition modifies genes or proteins that inhibit tumor suppression for example by inhibiting expression of those genes or proteins.

Another aspect of the invention provides methods of inhibiting tumorigenesis or growth of a tumor, such as melanoma, comprising administering to a patient suffering from a tumor a composition that modifies expression of a target gene, where the composition comprises a compound selected from the group consisting of an antisense oligonucleotide, an oligonucleotide that binds to mRNA to form a triplex, an RNAi molecule, a siRNA, an RNAi, an miRNA, a shRNA, or a nucleic acid molecule encoding a siRNA, an RNA, an miRNA, or a shRNA.

In another aspect, the invention provides methods of inhibiting tumor growth by administering to a patient suffering from a tumor a composition that modifies, for example by inhibiting, expression of a target gene, wherein the composition comprises a human antibody.

In another aspect, the invention provides methods of detecting a tumor, such as melanoma, or a pre-malignant transformation in a mammal, for example, in humans, using a kit, wherein the kit comprises primers or probe that specifically bind or hybridize, under stringent conditions, with nucleic acid molecules identified by the genes in Tables 1, 2 and/or 5.

In another aspect, the invention provides methods of detecting a tumor such as melanoma or a pre-malignant transformation in a mammal, for example, in humans, using a kit suitable for performing PCR, and wherein the kit comprises primers specific for the amplification of nucleic acid molecules identified by the genes in Tables 1, 2 and/or 5.

One aspect of the invention provides methods for detection of a tumor such as melanoma or a pre-malignant transformation in a mammal, comprising: a) assaying the level of expression of at least one of the oxidative stress pathway genes in a biological subject in a sample taken from a region of the mammal that is suspected to be precancerous or cancerous, or from a bodily fluid of the mammal, thereby generating data for a test level, where the gene is selected from the gene panel consisting of the genes listed in Tables 1, 2 and/or 5; and b) comparing the level of expression of the test gene to data for at least one control gene, wherein the expression level of the gene in the biological subject relative to the corresponding control indicates the presence of a tumor, such as melanoma, or a pre-malignant transformation in the mammal.

Another aspect of the invention provides methods for inhibiting a tumor, such as melanoma, by administering to a patient suffering from a tumor a composition that modifies expression of a gene or protein listed in Tables 1, 2 and/or 5, for example by inhibiting expression of the gene or protein or that inhibits activity of the protein.

Still another aspect of the invention provides methods for detection of a tumor, such as melanoma, or a pre-malignant transformation in a mammal, wherein the detection is carried out using a kit, wherein the kit comprises primers or probe that specifically bind or hybridize, under stringent condition, with nucleic acid molecules identified by the genes in Tables 1, 2 and/or 5. Furthermore, the detection is carried out using a kit suitable for performing PCR, where the kit contains primers specific for the amplification of one or more nucleic acid molecules identified by the genes in Tables 1, 2 and/or 5.

In yet another aspect, the invention provides serological tests for estimating the risk of developing a tumor, such as melanoma, or a pre-malignant transformation in an individual, comprising detecting the baseline status of a panel of the genes or their proteins listed in Tables 1, 2 and/or 5 in a bodily fluid, such as blood, which reflect a combination of inherited factors that come together to give a person more or less risk of developing cancer.

Another aspect of the invention provides methods for cutaneous biopsy test for estimating the risk of developing a tumor such as melanoma or a pre-malignant transformation in an individual, comprising detecting the baseline status of the gene or protein panel listed in Tables 1, 2 and/or 5 in normal tissues which reflect a combination of inherited factors that come together to give a person more or less risk of developing cancer.

In still another aspect, the invention provides methods for determining the efficacy of a therapeutic treatment regimen in a patient, comprising: a) measuring expression levels of one or more genes or proteins or activit(ies) of one or more proteins in a first biological sample obtained from the patient, thereby generating data for a test level, wherein the one or more genes or proteins are selected from the panel consisting of the genes listed in Tables 1, 2 and/or 5; b) administering the treatment regimen to the patient; measuring the expression or activity levels of the gene(s) or protein(s) in a second biological sample from the patient at a time following administration of the treatment regimen; and c) comparing the expression or activity levels of the gene(s) or protein(s) in the first and the second biological samples, wherein data showing decrease in the levels in the second biological sample relative to the first biological sample indicates that the treatment regimen is effective in the patient.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows a pathway schematic of gene products that respond to p16.

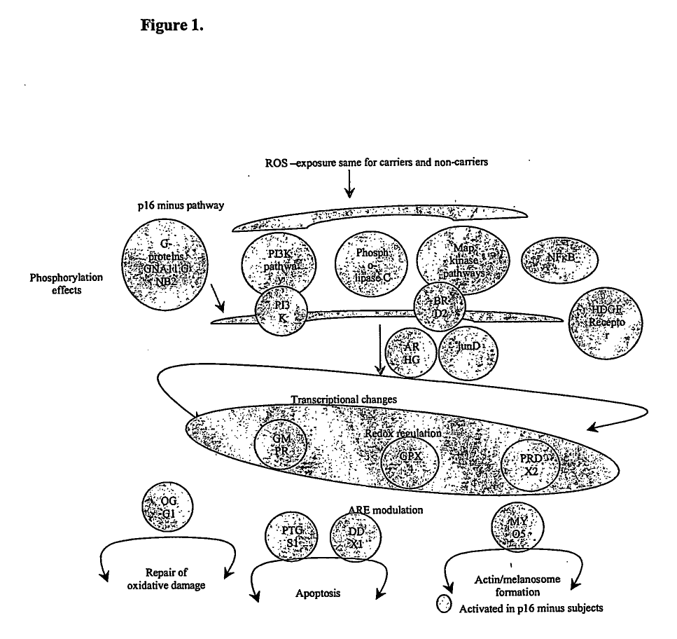

### DETAILED DESCRIPTION OF THE INVENTION

The invention provides new and improved methods for prediction, prevention, and treatment of tumors, such as melanoma and related malignancies. Genes having altered expression levels during pre-malignant transformation have been identified, and the changes in gene expression have been quantified. The relative changes in gene expression in nevi corresponding to mutation carriers and non-carriers and in those potentially cancerous or benign have been measured, and these measurements provide additional insight into the progress and development of, for example, melanoma. Moreover, by measuring changes in the expression of these genes or their protein products or by measuring changes in activity of the gene products, the risk of, for example, melanoma (or related malignancies) can be determined. In addition, these changes in gene expression or gene product expression/activity can be used to predict a patient's response to therapy and also permit the physician to measure the patient's response to therapy.

To identify genes whose expression, or inappropriate expression, is involved in the development of melanoma lesions, the expression profiles of normal and atypical nevi derived from patients that carry, and do not carry, a p16 mutation, may be compared. Germline p16 mutations predispose patients to the development of melanoma—as many as 25% of early sporadic melanomas demonstrate mutations in p16—and many more early melanomas demonstrate dysfunction of the p16 molecular pathway. The analysis of expression profiles from carriers and non-carriers of the p16 mutation reveals a dramatic pattern of gene expression that converges on oxidative stress pathways. The study of this pattern of differential expression allows identification of a novel molecular mechanism by which p16 mutations lead to increased susceptibility and progression to melanoma. This mechanism is broadly applicable to other tumor suppressor-mediated malignancies and is exceptionally well-suited as a source of diagnostic targets for early detection of cancer.

The present inventors have found early manifestations of increased susceptibility toward melanoma in increased baseline levels of expression (or activity) for some genes and decreased baseline levels of expression (or activity) for other genes. Some of the genes are involved in responding to oxidative stress, such as those encoding DNA excision repair enzymes, enzymes involved in lipid peroxidation, and genes that are secondarily activated during a period of increased oxidative stress. These genes are set forth in Tables 1, 2, and 5. Genes of particular interest are set forth in Table 5, and include SOD2, GPX4, PTGDS/PGDS2, CYP21A2, ACADM, AKR1C1, ACS3/FACL3, and KRT19.

In one embodiment, the expression of one or more of the genes set forth in Tables 1, 2, and/or 5 is monitored to detect and/or characterize a pre-malignant transformation. In other embodiments, multiple genes can be monitored, for example, 2, 3, 4, 5, 6, 7, 8, 9, 10, 15, 20, 50 etc. of these genes are monitored. In a desirable embodiment, the direction of change in the gene activity will be an increase, or decrease, as appropriate based on the direction (up or down) in microarray fold change measured by experimentation and reported in Tables 1, 2 and/or 5. It will be appreciated that, in the context of the present invention, a description of a change in gene expression can encompass not only changes at the nucleic acid level, but also at the level of production of the corresponding gene product(s) and also can encompass changes in activity of the gene product(s), unless otherwise indicated.

Other genes contemplated are involved in the cell cycle regulation pathway. Particularly desirable diagnostic genes from this pathway include, for example, CDC25A, BRD2, BCL10, JAK1, and FOXC1/FKHL7. In an embodiment, some or all of these genes are monitored. In a desirable embodiment, the direction of change in the gene activity will be an increase, or decrease, as appropriate based on the direction (up or down) in microarray fold change measured by experimentation and reported in Table 5.

Another desirable pathway is DNA repair. For example, MutS homolog 5 (MSH5) gene expression was found to be increased approximately two fold in carrier tissue versus non-carrier tissue.

In a desirable embodiment, at least two, three, four, five, six, seven, eight, nine, ten, eleven, twelve, thirteen, fourteen, fifteen, twenty, fifty, seventy five, one hundred, one hundred fifty or even more genes and/or expression products associated as prognostic indicators are monitored together to obtain improved prognostic reliability. The tests may be automated, or as simple as a histological stain/comparison. One or more genes or gene products listed on any of Tables 1-5 may be used.

In a particularly desirable embodiment, the expression of a gene listed in any of Tables 1, 2, and/or 5 is monitored simultaneously, or nearly simultaneously (i.e. by a different test but on a common biopsy) with that of another gene. Desirably, two, three or more genes listed on this table are assayed, for improved diagnostic value. For example, a biopsy of a suspicious nevus could be stained with a two or three color stain, with each color corresponding to a different labeled antibody that reacts with a separate gene product. All of the genes listed in the tables are contemplated for this use, although the 14 genes listed in Table 5, along with their direction of expression change (carrier/non-carrier) are particularly desirable. By assaying two, three or even more gene products, a greater confidence result may be obtained.

For each case, one or more gene copies, the level of gene expression or activity of protein produced by gene activity desirably may be assayed, according to an embodiment. For example, quantitative PCR and RT-PCR may be used to detect and quantitate genetic material. In this context, Table 5 shows RT-PCR measurements of gene expression of the genes SOD2, GPX4, PTGDS/PGDS2, CDC25A, BRD2, and MSH5 and these are particularly useful, and have demonstrated prognostic potential, as summarized in Table 5.

The present inventors' observations are consistent with prior observations that cell cycle regulatory proteins, such as p16, may act as checkpoint monitors that allow repair of oxidative damage to occur prior to cell division. If p16 mutation-carrying cells are slightly less competent in checkpoint function, cellular damage is likely to accumulate over time, leading to the need for (and reflexive) up-regulation of genes that manage this stress.

Definitions:

“Biological sample” as used herein refers to a sample obtained from a biological subject, including sample of biological tissue or fluid origin, obtained, reached, or collected in vivo or in situ, that contains or is suspected of containing nucleic acids listed in Table 1 and/or Table 2 or encoded polypeptides. A biological sample also includes samples from a region of a biological subject containing precancerous or cancer cells or tissues. Such samples can be, but are not limited to, organs, tissues, fractions and cells isolated from mammals including, humans such as a patient, horses, dogs, mice, and rats. Biological samples also may include sections of the biological sample including tissues, for example, frozen sections taken for histologic purposes.

“Providing a biological subject or sample” means to obtain a biological subject in vivo or in situ, including tissue or cell sample for use in the methods described in the present invention. Most often, this will be done by removing a sample of cells from an animal, but also can be accomplished in vivo or in situ or by using previously isolated cells (for example, isolated from another person, at another time, and/or for another purpose).

A “control sample” refers to a sample of biological material from one or more healthy, cancer-free subjects. Advantageously a control sample is from the same species as the biological sample under study. The expression level of any of the genes listed in Table 1 and/or Table 2 in a control sample advantageously is typical of the general population of normal, cancer subjects of the same species. This sample either can be collected from a healthy subject for use in the methods described herein, or it can be any biological material representative of normal, cancer-free animals suitable for use in those methods. A control sample also can be obtained from normal tissue from the animal that has cancer or is suspected of having cancer. A control sample also can refer to a given expression level of any of the genes listed in Table 1 and/or Table 2, representative of the cancer-free population, that has been previously established based on measurements from normal, cancer-free subjects. Alternatively, a biological control sample can refer to a sample that is obtained from a different individual or can be a normalized value based on baseline data obtained from a population. Further, a control sample can be defined by a specific age, sex, ethnicity or other demographic parameters. In some situations, the control is implicit in the particular measurement. An example of an implicit control is where a detection method can only detect expression level of any of the genes listed in Table 1 and/or Table 2 or the corresponding gene copy number, when a level higher than that typical of a normal, cancer-free subject is present. Another example is in the context of an immunohistochemical assay where the control level for the assay is known. Other instances of such controls are within the knowledge of the skilled person.

“Cancer” in a subject refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor, such cells may exist locally within a subject animal, or circulate in the blood stream as independent cells, for example, leukemic cells.

“Tumor” refers to all neoplastic cell growth and proliferation, whether malignant or benign, and all precancerous and cancerous cells and tissues.

“Precancerous” refers to cells or tissues having characteristics relating to changes that may lead to malignancy or a pre-malignant transformation or a cancer. Examples include adenomatous growths in tissues or conditions, for example, dysplastic nevus syndrome, a precursor to malignant melanoma of the skin. Examples also include, abnormal neoplastic, in addition to dysplastic nevus syndromes, polyposis syndromes, prostatic dysplasia, and other such neoplasms, whether the precancerous lesions are clinically identifiable or not.

A “target gene” refers to a differentially expressed gene in which modulation of the level of gene expression or of gene product activity prevents and/or ameliorates disease progression, for example, a tumor growth. Thus, compounds that modulate the expression of a target gene, the target genes, or the activity of a target gene product can be used in the diagnosis, treatment or prevention of a disease. In the context of the present invention, target genes include the genes listed in Table 1, Table 2, and/or Table 5 and their variants, as described herein. The skilled artisan will understand that the term “target gene” comprehends any splice variant of a gene.

“Gene expression” refers to the biosynthesis of a gene product. For example, in the case of a structural gene, gene expression involves transcription of the structural gene into mRNA and the translation of mRNA into one or more polypeptides.

The term “operably associated” is used to describe the connection between regulatory elements and a gene or its coding region. That is, gene expression is typically placed under the control of certain regulatory elements, including constitutive or inducible promoters, tissue-specific regulatory elements, and enhancers. Such a gene or coding region is the to be “operably linked to” or “operatively linked to” or “operably associated with” the regulatory elements, meaning that the gene or coding region is controlled or influenced by the regulatory element.

“Sequence homology” is used to describe the sequence relationships between two or more nucleic acids, polynucleotides, proteins, or polypeptides, and is understood in the context of and in conjunction with the terms including: (a) reference sequence, (b) comparison window, (c) sequence identity, (d) percentage of sequence identity, and (e) substantial identity or “homologous.”

(a) A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

(b) A “comparison window” includes reference to a contiguous and specified segment of a polynucleotide sequence, wherein the polynucleotide sequence may be compared to a reference sequence and wherein the portion of the polynucleotide sequence in the comparison window may comprise additions, substitutions, or deletions (i.e., gaps) compared to the reference sequence (which does not comprise additions, substitutions, or deletions) for optimal alignment of the two sequences. Generally, the comparison window is at least 20 contiguous nucleotides in length, and optionally can be 30, 40, 50, 100, or longer. Those of skill in the art understand that to avoid a misleadingly high similarity to a reference sequence due to inclusion of gaps in the polynucleotide sequence a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math., 2: 482, 1981; by the homology alignment algorithm of Needleman and Wunsch, J. Mol. Biol., 48: 443, 1970; by the search for similarity method of Pearson and Lipman, Proc. Natl. Acad. Sci. USA, 8: 2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73: 237-244, 1988; Corpet, et al., Nucleic Acids Research, 16:881-90, 1988; Huang, et al., Computer Applications in the Biosciences, 8:1-6, 1992; and Pearson, et al., Methods in Molecular Biology, 24:7-331, 1994. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Unless otherwise stated, sequence identity/similarity values provided herein refer to the value obtained using the BLAST 2.0 suite of programs, or their successors, using default parameters. Altschul et al., Nucleic Acids Res, 2:3389-3402, 1997. It is to be understood that default settings of these parameters can be readily changed as needed in the future.

As those ordinary skilled in the art will understand, BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163, 1993) and XNU (Claverie and States, Comput. Chem., 17:191-1, 1993) low-complexity filters can be employed alone or in combination.

(c) “Sequence identity” or “identity” in the context of two nucleic acid or polypeptide sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not deleteriously change the functional properties of the molecule. Where sequences differ in conservative substitutions, the percent sequence identity may be adjusted upwards to correct for the conservative nature of the substitution. Sequences which differ by such conservative substitutions are said to have sequence similarity. Approaches for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17, 1988, for example, as implemented in the program PC/GENE (Intelligenetics, Mountain View, Calif., USA).

(d) “Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) (i) The term “substantial identity” or “homologous” in their various grammatical forms means that a polynucleotide comprises a sequence that has a desired identity, for example, at least 60% identity, preferably at least 70% sequence identity, more preferably at least 80%, still more preferably at least 90% and even more preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, more preferably at least 70%, 80%, 90%, and even more preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, for example, when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid, although such cross-reactivity is not required for two polypeptides to be deemed substantially identical.

(e) (ii) The terms “substantial identity” or “homologous” in their various grammatical forms in the context of a peptide indicates that a peptide comprises a sequence that has a desired identity, for example, at least 60% identity, preferably at least 70% sequence identity to a reference sequence, more preferably 80%, still more preferably 85%, even more preferably at least 90% or 95% sequence identity to the reference sequence over a specified comparison window. Preferably, optimal alignment is conducted using the homology alignment algorithm of Needleman and Wunsch, J. Mol. Biol., 48:443, 1970. An indication that two peptide sequences are substantially identical is that one peptide is immunologically reactive with antibodies raised against the second peptide, although such cross-reactivity is not required for two polypeptides to be deemed substantially identical. Thus, a peptide is substantially identical to a second peptide, for example, where the two peptides differ only by a conservative substitution. Peptides which are “substantially similar” share sequences as noted above except that residue positions which are not identical may differ by conservative amino acid changes. Conservative substitutions typically include, but are not limited to, substitutions within the following groups: glycine and alanine; valine, isoleucine, and leucine; aspartic acid and glutamic acid; asparagine and glutamine; serine and threonine; lysine and arginine; and phenylalanine and tyrosine, and others as known to the skilled person.

“Antisense RNA”: In eukaryotes, RNA polymerase catalyzes the transcription of a structural gene to produce mRNA. A DNA molecule can be designed to contain an RNA polymerase template in which the RNA transcript has a sequence that is complementary to that of a preferred mRNA. The RNA transcript is termed an “antisense RNA.” Antisense RNA molecules can inhibit mRNA expression (for example, Rylova et al., Cancer Res, 62(3):801-8, 2002; Shim et al., Int. J. Cancer, 94(1):6-15, 2001). Antisense RNA also may be synthesized by chemical synthesis. Antisense RNA also encompasses synthetic molecules containing stabilized ribonucleotide analogs and/or ribonucleotide structures. Such analogs and structures are well known in the art.

“Antisense nucleic acid” “antisense DNA” or “DNA decoy” or “decoy molecule:” With respect to a first nucleic acid molecule, a second DNA molecule or a second chimeric nucleic acid molecule that is created with a sequence which is a complementary sequence or homologous to the complementary sequence of the first molecule or portions thereof is referred to as the antisense DNA or DNA decoy or decoy molecule of the first molecule. The term “decoy molecule” also includes a nucleic molecule, which may be single or double stranded, that comprises DNA or PNA (peptide nucleic acid) (Mischiati et al., Int. J. Mol. Med., 9(6):633-9, 2002), and that contains a sequence of a protein binding site, preferably a binding site for a regulatory protein and more preferably a binding site for a transcription factor. Applications of antisense nucleic acid molecules, including antisense DNA and decoy DNA molecules are known in the art, for example, Morishita et al., Ann. N Y Acad. Sci., 947:294-301, 2001; Andratschke et al., Anticancer Res, 21:(5)3541-3550, 2001.

“siRNA” refers to small interfering RNAs, which also include short hairpin RNA (“shRNA”) (Paddison et al., Genes & Dev. 16: 948-958, 2002), that are capable of causing interference (as described herein for RNAi) and can cause post-transcriptional silencing of specific genes in cells, for example, mammalian cells (including human cells) and in the body, for example, mammalian bodies (including humans). The phenomenon of RNA interference (RNAi) is described and discussed in Bass, Nature, 411:428-29, 2001; Elbashir et al., Nature, 411:494-98, 2001; and Fire et al., Nature, 391:806-11, 1998, wherein methods of making interfering RNA also are discussed. Exemplary siRNAs according to the invention could have up to 29 bps, 25 bps, 22 bps, 21 bps, 20 bps, 15 bps, 10 bps, 5 bps or any integer thereabout or therebetween.

“miRNA” refers to microRNA, a class of small RNA molecules or a small noncoding RNA molecules, that are capable of causing interference, inhibition of RNA translation into protein, and can cause post-transcriptional silencing of specific genes in cells, for example, mammalian cells (including human cells) and in the body, for example, mammalian bodies (including humans) (see, Zeng and Cullen, RNA, 9(1):112-123, 2003; Kidner and Martienssen Trends Genet, 19(1): 13-6, 2003; Dennis C, Nature, 420(6917):732, 2002; Couzin J, Science 298(5602):2296-7, 2002). Previously, the miRNAs were known as small temporal RNAs (stRNAs) belonged to a class of non-coding microRNAs, which have been shown to control gene expression either by repressing translation or by degrading the targeted mRNAs (see Couzin J, Science 298(5602):2296-7, 2002), which are generally 20-28 nt in length (see Finnegan et al., Curr Biol, 13(3):236-40, 2003; Ambros et al., RNA 9(3):277-279, 2003; Couzin J, Science 298(5602):2296-7, 2002). Unlike other RNAs (for example, siRNAs or shRNAs), miRNAs or stRNAs are not encoded by any microgenes, are generated from aberrant (probably double-stranded) RNAs by an enzyme called Dicer, which chops double-stranded RNA into little pieces (see Couzin J, Science 298(5602):2296-7, 2002). According to the invention, miRNA having different sequences but directed against genes listed in Table 1 and/or Table 2 can be administered concurrently or consecutively in any proportion, including equimolar proportions.

Stabilized RNA: A stabilized RNAi, siRNA, miRNA, or a shRNA as described herein, is protected against degradation by exonucleases, including RNase, for example, using a nucleotide analogue that is modified at the 3′ position of the ribose sugar (for example, by including a substituted or unsubstituted alkyl, alkoxy, alkenyl, alkenyloxy, alkynyl or alkynyloxy group as defined above). The RNAi, siRNA or a shRNA also can be stabilized against degradation at the 3′ end by exonucleases by including a 3′-3′-linked dinucleotide structure (Ortigao et al., Antisense Research and Development 2:129-146 (1992)) and/or two modified phospho bonds, such as two phosphorothioate bonds.

“Inhibitors” refers to molecules that inhibit and/or block an identified function. Any molecule having potential to inhibit and/or block an identified function can be a “test molecule,” as described herein. For example, referring to oncogenic function or anti-apoptotic activity of genes listed in Table 1 and/or Table 2, such molecules can be identified using in vitro and in vivo assays for genes listed in Table 1 and/or Table 2. Inhibitors are compounds that partially or totally block activities of any of the genes listed in Table 1 and/or Table 2, decrease, prevent, or delay their activation, or desensitize its cellular response. This can be accomplished by binding to expression product of any of the genes listed in Table 1 and/or Table 2 directly or via other intermediate molecules. An antagonist or an antibody that blocks activity of expression product of any of the genes listed in Table 1 and/or Table 2, including inhibition of oncogenic function or anti-apoptotic activity, is considered to be such an inhibitor. Inhibitors according to the instant invention is: a siRNA, an RNAi, a shRNA, an antisense RNA, an antisense DNA, a decoy molecule, a decoy DNA, a double stranded DNA, a single-stranded DNA, a complexed DNA, an encapsulated DNA, a viral DNA, a plasmid DNA, a naked RNA, an encapsulated RNA, a viral RNA, a double stranded RNA, a molecule capable of generating RNA interference, or combinations thereof. The group of inhibitors of this invention also includes molecules that are genetically modified, for example, versions with altered activity. The group thus is inclusive of the naturally occurring protein as well as synthetic ligands, antagonists, agonists, antibodies, small chemical molecules and the like.

An “aptamer” is a peptide, a peptide-like, a nucleic acid, or a nucleic acid-like molecule that is capable of binding to a specific molecule (for example, genes listed in Table 1 and/or Table 2) of interest with high affinity and specificity. An aptamer also can be a peptide or a nucleic acid molecule that mimics the three dimensional structure of active portions of the peptides or the nucleic acid molecules of the invention. (see, for example, James W., Current Opinion in Pharmacology, 1:540-546 (2001); Colas et al., Nature 380:548-550 (1996); Tuerk and Gold, Science 249:505 (1990); Ellington and Szostak, Nature 346:818 (1990)). The specific binding molecule of the invention may be a chemical mimetic; for example, a synthetic peptide aptamer or peptidomimetic. It is preferably a short oligomer selected for binding affinity and bioavailability (for example, passage across the plasma and nuclear membranes, resistance to hydrolysis of oligomeric linkages, adsorbance into cellular tissue, and resistance to metabolic breakdown). The chemical mimetic may be chemically synthesized with at least one non-natural analog of a nucleoside or amino acid (for example, modified base or ribose, designer or non-classical amino acid, D or L optical isomer). Modification also may take the form of acylation, glycosylation, methylation, phosphorylation, sulfation, or combinations thereof. Oligomeric linkages may be phosphodiester or peptide bonds; linkages comprised of a phosphorus, nitrogen, sulfur, oxygen, or carbon atom (for example, phosphorothionate, disulfide, lactam, or lactone bond); or combinations thereof. The chemical mimetic may have significant secondary structure (for example, a ribozyme) or be constrained (for example, a cyclic peptide).

A peptide aptamer is a polypeptide or a polypeptide-like molecule that is capable of binding to a specific molecule (for example, peptides encoded by genes listed in Table 1 and/or Table 2) of interest with high affinity and specificity. A peptide aptamer also can be a polypeptide molecule that mimics the three dimensional structure of active portions of the polypeptide molecules of the invention. A peptide-aptamer can be designed to mimic the recognition function of complementarity determining regions of immunoglobulins, for example. The aptamer can recognize different epitopes on the protein surface (for example, proteins encoded by genes listed in Table 1 and/or Table 2) with dissociation equilibrium constants in the nanomolar range; those inhibit the protein (for example, proteins encoded by genes listed in Table 1 and/or Table 2) activity. Peptide aptamers are analogous to monoclonal antibodies, with the advantages that they can be isolated together with their coding genes, that their small size facilitates solution of their structures, and that they can be designed to function inside cells.

A peptide aptamer is typically between about 3 and about 100 amino acids or the like in length. More commonly, an aptamer is between about 10 and about 35 amino acids or the like in length. Peptide-aptamers may be prepared by any known method, including synthetic, recombinant, and purification methods (James W., Current Opinion in Pharmacology, 1:540-546 (2001); Colas et al., Nature 380:548-550 (1996)).

A nucleic acid aptamer is a nucleic acid or a nucleic acid-like molecule that is capable of binding to a specific molecule (for example, genes listed in Table 1 and/or Table 2) of interest with high affinity and specificity. A nucleic acid aptamer also can be a nucleic acid molecule that mimics the three dimensional structure of active portions of the nucleic acid molecules of the invention. A nucleic acid-aptamer is typically between about 9 and about 300 nucleotides or the like in length. More commonly, an aptamer is between about 30 and about 100 nucleotides or the like in length. Nucleic acid-aptamers can be prepared by any known method, including synthetic, recombinant, and purification methods (James W., Current Opinion in Pharmacology, 1:540-546 (2001); Colas et al., Nature 380:548-550 (1996)).

Determination of Changes in Gene Regulation in Melanoma and Pre-Melanoma

It previously was known that oxidative damage to DNA is one mechanism by which mutations can occur and lead to carcinogenesis. However, prior workers have focused on the causal relationship between oxidative damage and carcinogenesis rather than evaluating expression changes in these genes as an early marker for pre-malignant transformation. The present inventors have identified a panel of genes and gene products related to the oxidative stress pathway that serve as markers of early detection of melanoma, and that also serve as early markers of transformation of other neoplasms, and that provide a mechanism by which patients can be screened for cancer risk.

Experimental design: Gene expression patterns in nevi from one p16 mutation carrier and one non-carrier using cDNA microarrays were evaluated. At the biological treatment level, the design involves two classification factors, the p16 carrier status and the nevus morphology. Data from an initial block of this experimental design (one carrier and one non-carrier of the p16 mutation each with an atypical and benign nevus −4 treatments total) were analyzed.

Differential Analysis of Gene Expression: Analysis at the gene level involves estimation of (i) effect of carrier status on expression (mutation effect) and (ii) effect of nevus morphology—atypical versus benign (nevus effect). The mutation effect clearly distinguishes whether the differential expression of a gene is associated with the presence of a p16 mutation. The nevus effect describes whether a gene is differentially expressed in an atypical mole versus a benign mole. The interactions between the mutation and nevus effects are also important as they tend to indicate genes that are differentially expressed in atypical nevi but selectively for carriers of the mutation.

The mutation effect was selected to be the primary screen for identifying genes of interest. The genes that were identified in the primary screen were then further characterized those genes for effects on nevi and other functions.

Characterizing Response Pathways from Expression Patterns:

Following the identification of the genes of interest from the initial screen, their pathway functionality is studied to further characterize genes of high interest in terms of their functional annotations and sub-cellular localization. This process is applied to gene clusters with high mutation effects using annotations publicly available in Gene Ontology and employing the informatics algorithms developed by Silico Insights (Massachusetts). Initial characterization of genes is carried out using a filtering criterion on the size of the mutation effect In the results described in Table 2, the criterion used was mutation effect >1.5 and <−1.5 (i.e. those genes over- or under-expressed differentially by 50% or more in carriers versus non-carriers of p16 mutation), though the skilled artisan will recognize that other criteria may be used. The annotation reports of the genes meeting the criterion are shown in Table 3. Based on membership in pre-dominant functions defining the high differential expression clusters (Table 4), genes are screened for statistical significance at a defined confidence level, for example at the 95% confidence level, based on a predefined number of replicates. The results of these analyses are used to compile a list of potential target genes. The final list compiled using the criteria set forth above is shown in Table 1.

This analysis reveals a panel of genes involved in the response to oxidative stress. Thus, OGG1 (8-oxoguanine DNA glycosylase) is a DNA mismatch repair enzyme that assists in repair of damaged DNA. Similarly, peroxiredoxin 2 and glutathione peroxidase 4 are important molecules in the lipid peroxidation pathway. Down regulation of prostaglandin-endoperoxide synthase 1 (COX1) is consistent with a need to decrease arachidonate-pathway lipids and is analogous to a self-induced anti-inflammatory response similar to that seen with COX1 and COX2 inhibitors. It is well established that guanine nucleotide binding proteins (G proteins), particularly those of the Gq and Rho subfamily, mediate signal transduction of bioactive lipid mediators such as lysophosphatidic acid (LPA) and sphingolipids. Thus, the differentially activated G proteins observed here likely mediate the signal transduction of the elevated oxidative damage (Gq alpha 11, G beta 2, and RhoG).

Further evidence of increased G protein metabolism is demonstrated by the upregulation of guanosine monophosphate reductase. Bromodomain-containing 2 protein (Ring 3) is a nuclear mitogen activated kinase, involved in signal transduction and likely is involved in transmission of the G protein mediated responses we are observing. It is known that oxidative insults activate the transcription of genes in part by the AP1 transcription factor. Increased expression of junD, a component of the AP1 family of transcription factors, is consistent with increased transmission of oxidative damage to the nucleus. Three other transcription factors appear to be involved in the stress response here: hepatoma-derived growth factor, nuclear factor I/C, and DEAD/H box polypeptide 1. Phosphomevalonate kinase is a key regulatory enzyme in the biosynthesis of sterols and isoprenoids and may be activated in response to an increased need to replace sterol-containing molecules or isoprenes such as the fat soluble vitamins or carotenoids.

Determination of Reference Level: The reference level used in the methods of the present invention is the level of gene expression in relatively healthy tissue. This may mean the level of gene expression in a control sample, or it may mean the level of gene expression prior to the development of melanoma. The reference level may be determined from global values assayed from healthy individuals.

Practical and Commercial Applications: The panel of genes that have been identified can be utilized for one or more of the applications discussed below (a-g). The methods that may be used to examine differential expression and regulation of this panel of genes include sensitive and quantitative techniques such as: 1) mRNA detection methods (e.g. RT-PCR, Northern dot/slot blot); 2) protein expression (e.g. Western, ELISA), 3) post-translational protein modification (2-D electrophoresis: kinase, phosphorylation, glycosylation, and prenylation assays), and 4) other biochemical assays designed to detect specific enzymatic activities of selected members of the gene panel.

a. Diagnostic marker/s for nevi and early melanoma—because many of the proteins described are enzymes, and because the pre-malignant lesions in question are on the surface of the skin, non-invasive assays can be used to measure these enzymes (i.e. provide substrate and detect product in vivo).

b. Prognostic indicator for melanoma—the degree to which the genes and their protein products are differentially regulated tends to reflect the degree to which the cells have become genomically unstable. Accordingly, the degree of changes in regulation also correlates with the aggressiveness (and outcome) of the melanoma (or other neoplasm).

c. Serological or cutaneous biopsy test for cancer/melanoma susceptibility—the baseline status of the gene (and corresponding protein product) panel in normal tissues or in blood can reflect a combination of inherited factors (susceptibility genes or modifier genes) that come together to give a person more or less risk of developing cancer. Those at highest risk would benefit from chemoprevention strategies (see below) since it would then be possible to intervene before transformation occurs.

d. Mechanism of evaluating effectiveness of chemoprevention strategies—in order to evaluate any chemoprevention strategy, a biological endpoint is very useful. The panel of genes and their protein products described herein is useful in evaluating whether chemoprevention strategies have an impact.

e. Mechanism of evaluating effectiveness of p16-, melanoma, and cancer-directed therapies—in order to determine whether therapeutic regimens have been effective, it is important to be able to identify markers of reduced tumor burden as well as markers of early recurrence. This panel of genes and their protein products serves as a highly sensitive marker for treatment efficacy and early detection of recurrence.

f. Therapeutic—the molecular pathways identified herein participate in the tumor's ability to escape immune surveillance and chemotherapeutic regimens. Drugs that target these pathways enhance tumor sensitivity to treatment or synergize with specific treatment regimens. For example, if increased oxidative repair is essential for pre-malignant cells to remain under relatively normal control, targeting of one of the essential components of that pathway can enhance sensitivity to pro-apoptotic chemotherapeutic agents.

g. Cosmetic and anti-aging—Chemoprevention and anti-aging pathways overlap significantly, especially in the skin Genes that serve to respond and improve cellular responses to oxidative damage (identified serendipitously in this model) are effective anti-aging or chemopreventative agents when applied exogenously.

The genes listed in Table 1 and Table 2 and their respective proteins represent novel factors that have not been previously been demonstrated to play a role in the development of melanoma, specifically p16-dependent melanoma. The differential expression of these genes at baseline (as opposed to differential regulation in response to a drug or environmental stimulus; these RNAs were isolated from normal, untreated tissues) suggests that these changes represent an overall shift in the equilibrium of the expression of these genes. Study of their interactions with known proteins reveals network effects that connect key pathways in transcription, cell signaling by G-proteins, and oxidative stress and repair.

Without being bound by any theory, the inventors believe that an analysis of these genes and their proteins demonstrates the following pathways: 1) a baseline increase in products of oxidative damage (primarily DNA damage and lipid oxidation) occurs in cells with decreased capacity for checkpoint control, such as those with p16 mutations; 2) the increase in these damaged intracellular components serves as a stimulus to activate oxidative damage detection, signal transduction and repair mechanisms; 3) since the baseline level of damage in these cells is higher, and likely continues to accumulate over time, the intracellular equilibrium between damage and damage response genes is shifted in the pre-malignant cells relative to normal cells.

Two categories of application arise from the availability of these genes and proteins and their relationship with p16-susceptible melanoma.

Diagnostic/Prognostic strategies: The identification of highly differential molecular behavior related to these targets offers a novel strategy to develop an early indicator of melanoma susceptibility. Although the presence of p16 mutation itself makes an individual high risk within a familial context, measurement of products downstream of p16 action provides considerably higher reliability in prognosis. The selected factors for measurement based on one or more of these genes depends on the following criteria:

a. Extent of contribution of the gene or protein and change in its action to disease occurrence;

b. Ease of measurement of the molecular process—criteria will prioritize by enzymatic activity, extra-cellular presence and specificity to disease phenotype; and

c. Combination of one or more genes and proteins that make prognosis significantly more predictable.

Therapeutic strategies: The function of the genes described herein and their relationships offers new ways to construct and validate a mechanism that leads to melanoma in p16(−) individuals. The pathway identified from these genes has been described above.

The availability of a validated pathway based on key differential genes and proteins offers the opportunity to treat melanoma based on possible modulation of high contributory factors. The criteria for selecting a suitable target gene of genes for therapeutic intervention include:
a. Level of contribution to disease response;
b. Ease with which gene action (at RNA, protein modification or interaction levels) can be modified by external factors;
c. Centrality of the identified gene and/or its action that can affect other constitutive pathways deleteriously; and
d. Initial experimental verification of modulation resulting in change of disease response (using cultures of high-purity cell lines).

The selection of targets for diagnostic and therapeutic strategies may be different since factors amenable to easy measurement and easy modulation can be quite different Validation of the selection of a therapeutic target for application can be performed by (i) initial experiments in cell lines cultured from melanocytes where tumor response is arrested, and (ii) animal knock-outs where genetic modulation or loss of targets verify absence of disease phenotype in animals with the melanoma response.

The change in expression of certain of the identified genes is predictive, not just of the risk for melanoma itself, but is diagnostic of the stage of development of the disease. By identifying a set of genes whose expression changes during the development of melanoma, the inventors have shown that analysis of a greater numbers genes leads to a greater ability to predict the development of melanoma, and to determine the probability of its development In view of the importance that the identified genes may play in the etiology of melanoma, an ability to manipulate the expression of those genes or the translation of their proteins or those genes or proteins that regulate their activity is efficacious in the treatment of melanoma. Methods to treat melanoma may include gene therapy to increase the expression of genes down-regulated during the disease. Treatment may also include methods to decrease the expression of genes up-regulated during melanoma. Treatment to decrease gene expression may include, but is not limited to, the expression of anti-sense mRNA, siRNA, triplex formation or inhibition by co-expression. Furthermore, treatment alternatives may include methods to modify activity of the proteins of these gene products.

Identification of genes involved in the development of melanoma also makes possible an identification of proteins that affect the development of melanoma. Identification of such proteins makes possible the use of methods to affect their expression or alter their metabolism. Methods to alter the effect of expressed proteins include, but are not limited to, the use of specific antibodies or antibody fragments that bind the identified proteins, specific receptors that bind the identified protein, or other ligands or small molecules that inhibit the identified protein from affecting its physiological target and exerting its metabolic and biologic effects. In addition, those proteins that are down-regulated during the course of melanoma may be supplemented exogenously to ameliorate their decreased synthesis.

The identification of genes involved in the development of melanoma makes possible the prophylactic use of methods to affect gene expression or protein function, and such methods may be used to treat individuals at risk for the development of melanoma.

Elucidation of Changes in Gene Expression in Melanoma

The present inventors have identified the genes that undergo changes in expression during the development of melanoma. Those genes are listed in Table 2. The inventors have carried out this analysis using nucleic acid array analysis of tissue from patients as described in more detail below.

Now that a set of genes that undergo changes in expression during the development of melanoma, it is possible to predict the risk of melanoma by studying the changes of a smaller subset of those genes. Thus, although about 85 genes have been shown to have significant altered expression in melanoma, it is possible to reliably predict the risk of melanoma by analyzing one of these genes or a subset of these genes, for example a subset that contains two or three key members. In other embodiments, combinations of more genes and their proteins may be studied or, if desired, all or most of the genes listed in Table 1 and/or Table 2 can be studied. By measuring changes in expression of a set of genes and those of their corresponding proteins, rather than of a single gene or protein, the present invention provides increased statistical confidence that the changes observed are predictive of melanoma or the risk of developing melanoma- i.e., this provides reliable risk profiling of an individual. Thus, in some cases a change in expression of a single gene or protein may not increase susceptibility to disease sufficiently to cross the threshold for disease development. On the other hand, coordinated changes in expression of multiple specified genes, is much more likely to increase the risk of melanoma.

By assaying gene expression that influences expression of these genes it is possible to predict the risk of melanoma early in the development of the disease, or even prior to the development of clinically detectable melanoma. Such early prediction provides the clinician with opportunities to slow or halt the melanoma. Moreover, the invention provides new compositions that can be used to inhibit, slow, or prevent melanoma.

Dysregulation of Multiple Genes that Increase Susceptibility to Melanoma

Differential expression of genes can be measured directly in patient samples. Advantageously the samples used for the present invention are from skin tissue, especially from tissue that is suspected to be cancerous or pre-cancerous. The present inventors used nucleic acid array methods to identify those genes that exhibit significantly changed expression in tissues that is cancerous or that is predisposed to be cancerous. However, other methods for measuring changes in gene or protein expression are well known in the art. For example, levels of proteins can be measured in tissue sample isolates using quantitative immunoassays such as the ELISA. Kits for measuring levels of many proteins using ELISA methods are commercially available from suppliers such as R&D Systems (Minneapolis, Minn.) and ELISA methods also can be developed using well known techniques. See for example Antibodies: A Laboratory Manual (Harlow and Lane Eds. Cold Spring Harbor Press). Antibodies for use in such ELISA methods either are commercially available or may be prepared using well known methods. For proteins having enzymatic activity, protein levels may be measured using assays of enzymatic activity. Such assays are well known in the art.

Other methods of quantitative analysis of multiple proteins include, for example, proteomics technologies such as isotope coded affinity tag reagents, MALDI TOF/TOF tandem mass spectrometry, and 2D-gel/mass spectrometry technologies. These technologies are commercially available from, for example, Large Scale Proteomics Inc. (Germantown, Md.) and Oxford Glycosystems (Oxford UK).

Alternatively, quantitative mRNA amplification methods, such as quantitative RT-PCR, can be used to measure changes in gene expression at the message level. Systems for carrying out these methods also are commercially available, for example the TaqMan system (Roche Molecular System, Alameda, Calif.) and the Light Cycler system (Roche Diagnostics, Indianapolis, Ind.). Methods for devising appropriate primers for use in RT-PCR and related methods are well known in the art. In particular, a number of software packages are commercially available for devising PCR primer sequences.

Nucleic acid arrays offer are a particularly attractive method for studying the expression of multiple genes. In particular, arrays provide a method of simultaneously assaying expression of a large number of genes. Such methods are now well known in the art and commercial systems are available from, for example, Affymetrix (Santa Clara, Calif.), Incyte (Palo Alto, Calif.), Research Genetics (Huntsville, Ala.) and Agilent (Palo Alto, Calif.). See also U.S. Pat. Nos. 5,445,934, 5,700,637, 6,080,585, 6,261,776 the contents of which are hereby incorporated by reference in their entirety.

To study a set of genes having altered expression in melanoma using nucleic acid arrays, samples of total RNA or mRNA are obtained from patient tissue, and analyzed using methods that are well known in the art. Thus, for example, samples of suspect tissue can be obtained by biopsy. Total RNA can be obtained using commercially available kits, such as Triazol reagent (Invitrogen, Carlsbad, Calif.) and mRNA can be obtained from this sample by chromatography on oligo(dT) cellulose. The RNA is reverse transcribed and the resulting cDNA subjected to an amplification step. In one embodiment, the amplification is a linear RNA amplification method such as that described in U.S. Pat. Nos. 5,716,785 and 5,891 ,636, which are hereby incorporated by reference in their entirety. Detailed instructions for preparing amplified RNA are available, for example, in the manufacturer's directions for preparing samples for assay using the Affymetrix GeneChip system.

Once suitable nucleic acid samples have been obtained, the gene expression profiles are determined using the nucleic acid arrays according to the manufacturer's instructions. For every gene probe on the array this provides a quantitative gene expression level in the sample. The expression level for each gene can then be compared to a baseline value to determine whether expression has been altered. Thus, the gene expression level of genes in tissue under study can be compared to reference levels of those genes in healthy tissue where melanoma is not occurring. Preferably, those reference levels are obtained from the same subject, although it is possible to use reference levels from different subjects. In such cases it is preferred to use reference levels from subjects that resemble the test subject as closely as possible, for example in demographic criteria such as age, gender, ethnicity, etc.

Although it is possible to measure absolute gene expression levels, it often is more convenient to measure relative gene expression levels. Thus, levels of expression of a particular gene on the array are compared to a reference gene on the same array whose expression is known to be unaffected in melanoma, for example, a gene not shown in Tables 1, 2 or 5. This provides an internal control mechanism for the array and reduces any differences in results that are due to variability in the array, assay conditions, etc.

In each case, the level of gene expression is compared to a suitable baseline level of expression. The baseline level of expression can be the level found in healthy vascular tissue, a global concentration assayed from a pool of healthy individuals or some other objective baseline.

Thus, in one embodiment, the invention provides methods for assaying expression of more than one gene or protein selected from Tables 1, 2 and/or 5. The genes can be selected in combinations such that (i) increased expression of all targets (or markers) indicates melanoma; (ii) decreased expression of all targets indicates melanoma; (iii) decreased expression of some targets combined with increased expression of the remaining selected targets indicates melanoma. Regardless of the number of genes or proteins in the subset of analyzed genes, the expression profile satisfies the criteria to diagnose the disease set out above when (i) the expression of some genes is increased throughout the course of the disease; (ii) the expression of some genes is decreased throughout the course of the disease; (iii) expression of some of the genes are increased while others are decreased, or (iv) the expression of some genes is altered during the development of the disease.

The skilled artisan will recognize that, due to the heterogeneous nature of melanoma, not all individuals with melanoma will exhibit altered expression of every one of the genes listed in Tables 1, 2 and/or 5. Thus, it is possible that one, or a few genes or proteins will not exhibit significantly altered expression, and that different individuals will exhibit different levels of gene expression, yet, the coordinated changes in the expression of the totality of genes and proteins are highly predictive of the presence of or development of melanoma.

In general, where the expression of only a relatively small number of genes is studied, changes in expression in most or all of the genes provides a reliable diagnosis of melanoma. For example, where only three genes are measured, changes in expression of all three genes provides a reliable diagnosis of melanoma. Where five genes are studied, changes in three or four genes typically will provide a reliable diagnosis. In general, as the number of altered genes and proteins defining the diagnosis increases, it is possible to provide a reliable diagnosis by observing coordinated changes in expression of some or all factors together.

Methods of Studying Gene Expression of the Genes Listed in Tables 1, 2 and 5

Gene expression may be studied at the nucleic acid (RNA) level or the protein level. While each cell nucleus carries a complete set of genes only those genes expressed in each cell are transcribed into mRNA, which is then translated into proteins. Consequently, gene expression is tissue or even cell specific. Generally, it is thought that the greater the number of RNA molecules transcribed the greater the number of protein molecules translated from them and, accordingly, the results obtained using RNA or protein analysis should be the same, at least in terms of relative changes in levels of gene expression. An analysis of gene expression may therefore be directed at the quantity of a particular mRNA transcript or the amount of protein translated from it

RNA Expression

Methods of isolating RNA from tissue are well known in the art. See, for example, Sambrook et al. Molecular Cloning: A Laboratory Manual (Third Edition) Cold Spring Harbor Press, 2001. Commercial reagents also are available for isolating RNA.

Briefly, for example, cells or tissue are lysed and the lysed cells centrifuged to remove the nuclear pellet The supernatant is then recovered and the nucleic acid extracted using phenol/chloroform extraction followed by ethanol precipitation. This provides total RNA, which can be quantified by measurement of optical density at 260-280 nM.

mRNA can be isolated from total RNA by exploiting the “PolyA” tail of mRNA by use of several commercially available kits. QIAGEN mRNA Midi kit (Cat. No. 70042); Promega PolyATtract® mRNA Isolation Systems (Cat. No. Z5200). The QIAGEN kit provides a spin column using Oligotex Resin designed for the isolation of poly A mRNA and yields essentially pure mRNA from total RNA within 30 minutes. The Promega system uses a biotinylated oligo dT probe to hybridize to the mRNA poly A tail and requires about 45 minutes to isolate pure mRNA.

mRNA can also be isolated by using the cesium chloride cushion gradient method. Briefly the flash frozen tissue is homogenized in guanidinium isothiocyanate, layered over a cushion of cesium chloride and ultracentrifuged for 24 hours to obtain the total RNA.

Genetic Microarray Analysis

Microarray technology is an extremely powerful method for assaying the expression of multiple genes in a single sample of mRNA. For example, Gene Chip® technology commercially available from Affymetrix Inc. (Santa Clara, Calif.) uses a chip that is that is plated with probes for over thousands of known genes and expressed sequence tags (ESTs). Biotinylated cRNA (linearly amplified RNA) is prepared and hybridized to the probes on the chip. Complementary sequences are then visualized and the intensity of the signal is commensurate with the number of copies of mRNA expressed by the gene. In the data shown in Tables 1 and 2 the microarrays were from Research Genetics (Huntsville, Ala.) and references to vendor clone designations refer to Research Genetics' designations.

Quantitative PCR

Quantitative PCR (qPCR) employs the co-amplification of a target sequence with serial dilutions of a reference template. By interpolating the product of the target amplification with that a curve derived from the reference dilutions an estimate of the concentration of the target sequence may be made. Quantitative reverse transcription PCR (RTPCR) may be carried out on mRNA using kits and methods that are commercially available from, for example, Applied BioSystems (Foster City, Calif.) and Stratagene (La Jolla, Calif.) See also Kochanowsi, Quantitative PCR Protocols” Humana Press, 1999. For example, total RNA may be reverse transcribed using random hexamers and the TaqMan Reverse Transcription Reagents Kit (Perkin Elmer) following the manufacturer's protocols. The cDNA is amplified using TaqMan PCR master mix containing AmpErase UNG dNTP, AmpliTaq Gold, primers and TaqMan probe according to the manufacture's protocols. The TaqMan probe is target-gene sequence specific and is labeled with a fluorescent reporter (FAM) at the 5′ end and a quencher (e.g. TAMRA) at the 3′ end. Standard curves for both endogenous control and the target gene may be constructed and the comparison of the ration of CT (threshold cycle number) of target gene to control in treated and untreated cells is determined. This technique has been widely used to characterize gene expression.

Protein Expression

Gene expression may also be studied at the protein level. Target tissue is first isolated and then total protein is extracted by well known methods. Quantitative analysis is achieved, for example, using ELISA methods employing a pair of antibodies specific to the target protein.

A subset of the proteins listed in Table 1, Table 2 and/or Table 5 is soluble or secreted. In such instances the proteins may be found in a bodily fluid, such as the blood, serum, plasma, lymph and/or urine and an analysis of those proteins may be afforded by any of those methods described for the analysis of proteins in such fluids. This provides a minimally invasive means of obtaining patient samples for estimate of risk of developing tumor, such as melanoma. Methods for identifying secreted proteins are known in the art.

Treatment of Melanoma

The identification of the set of genes or proteins having altered expression during the development of melanoma provides new opportunities to treat melanoma. Identification of genes up-regulated in melanoma or pre-melanoma affords the ability to use methods to negatively affect their transcription or translation. Similarly, the identification of genes that are down-regulated during the development of melanoma affords the ability to positively affect their expression. Finally, the determination of the proteins encoded by these genes allows for the use of appropriate methods to ameliorate or potentiate the protein activities, which thereby could influence the development of melanoma.

Methods of Modifying Gene Expression

The present invention affords an ability to negatively affect the expression of genes that are up-regulated during the development of melanoma. Methods for down regulating genes are well known. It has been shown that antisense RNA introduced into a cell will bind to a complementary mRNA and thus inhibit the translation of that molecule. In a similar manner, antisense single stranded cDNA may be introduced into a cell with the same result. Further, co-suppression of genes by homologous transgenes may be effected because the ectopically integrated sequences impair the expression of the endogenous genes (Cogoni et al. Antonie van Leeuwenhoek, 1994; 65(3):205-9), and may also result in the transcription of antisense RNA (Hamada and Spanu, Mol. Gen Genet 1998). Methods of using short interfering RNA (RNAi) to specifically inhibit gene expression in eukaryotic cells have recently been described. See Tuschl et al., Nature 411:494-498 (2001).

In addition, stable triple-helical structures can be formed by bonding of oligodeoxyribonucleotides (ODNs) to polypurine tracts of double stranded DNA. (See, for example, Rininsland, Proc. Nat'l Acad. Sci. USA 94:5854-5859 (1997). Triplex formation can inhibit DNA replication by inhibition of transcription of elongation and is a very stable molecule.

Methods to Modify the Activity of Specific Proteins

When a specific protein has been implicated in the melanoma development pathway its activity can be altered by several methods. First, specific antibodies may be used to bind the protein thereby blocking its activity. Such antibodies may be obtained through the use of conventional hybridoma technology or may be isolated from libraries commercially available from Dyax (Cambridge, Mass.), MorphoSys (Martinsried, Germany), Biosite (San Diego, Calif.) and Cambridge Antibody Technology (Cambridge, UK). In addition, proteins usually exert their cellular effects by ligating to cellular receptors. Identification of the receptors to which proteins, which are implicated by the current invention as contributing to melanoma, bind will allow the design of specific ligand antagonists that block pathways mediating the effects leading to the development of melanoma.

The identification of genes that are down regulated during the development of melanoma leads to the ability to supplement their corresponding down-regulated proteins, thereby ameliorating the effect of their decreased synthesis.

The methods of the present invention may be used prophylactically to prevent the development of melanoma in at risk individuals.

The invention also provides aptamers of peptides encoded by genes listed in Table 1 and/or Table 2. In one aspect, the invention provides aptamers of isolated polypeptides comprising at least one active fragment having substantially homologous sequence of peptides encoded by genes listed in Table 1 and/or Table 2. The instant aptamers are peptide molecules that are capable of binding to a protein or other molecule, or mimic the three-dimensional structure of the active portion of the proteins encoded by the genes of Table 1 and/or Table 2.

According to one aspect of the invention, aptamers of the instant invention include non-modified or chemically modified RNA, DNA, PNA or polynucleotides. The method of selection can be by, but is not limited to, affinity chromatography and the method of amplification by reverse transcription ( or polymerase chain reaction (PCR). Such nucleic acid aptamers have specific binding regions which are capable of forming complexes with an intended target molecule in an environment wherein other substances in the same environment are not complexed to the nucleic acid.

The instant invention also provides aptamers of polynucleotides of genes listed in Table 1 and/or Table 2, or any fragment thereof. In another aspect, the invention provides aptamers of isolated polynucleotides comprising at least one active fragment having substantially homologous sequence of polynucleotides of genes listed in Table 1 and/or Table 2, or any fragment thereof). The instant aptamers are nucleic acid molecules that are capable of binding to a nucleic acid or other molecule, or mimic the three-dimensional structure of the active portion of the nucleic acids of the invention.

The invention also provides nucleic acids (for example, mRNA molecules) that include an aptamer as well as a coding region for a regulatory polypeptide. The aptamer is positioned in the nucleic acid molecule such that binding of a ligand to the aptamer prevents translation of the regulatory polypeptide.

The present invention, thus generally described, will be understood more readily by reference to the following examples, which are provided by way of illustration and are not intended to limit the invention.

### EXAMPLE 1

### Experimental Design and Statistical Analysis

One of the main concerns with cDNA microarray experiments is to minimize the dye-specific biases that could be introduced while at the same time taking advantage of the blocked nature of the cDNA microarray experiment to maximize the precision of the estimated effects. The experimental design involves two key factors, p16 mutation carrier status and nevus morphology, each factor having two different levels. In this scheme, multiple carriers and non-carriers of the mutation participate in the study and one or more benign and atypical nevi is extracted from each patient One or more independent RNA samples is extracted from each nevus.

Array background correction is applied for each channel (dye) separately to correct the signal intensities for potential systematic artifacts. In the correction method used in this analysis, the signals from each slide are sorted in increasing order and the 5 smallest signals are averaged to give the average background intensity for the array. This intensity is then subtracted from all signals. Those spots having lower intensity than the background are eliminated from further calculations. After correction for background, the (positive) signals are transformed using a Log₂ transformation.

Before analysis, the individual spot intensities are corrected for systematic biases of the experimental protocol such as array-to-array (hybridization) variability and dye effects. A linear model approach, similar to the one proposed by Wolfinger et al. (J Comput Biol 8:6 625-37 (2001)) is used to account for such systematic effects.

### EXAMPLE 2

### Confirmation by RT-PCR

Microarray experiments were repeated to re-evaluate the expression level differences between control (normal) tissues and nevus samples. From this work, 14 genes that particularly relate to premalignant transformation were found. These 14 genes, with their listed microarray fold change values, are summarized on Table 5. As seen in this table, some genes increased expression in correlation to premalignant transformation and some genes decreased expression.

Of the 14 genes listed in Table 5, SOD2 was studied in more detail. An antibody-based immunohistochemistry test was carried out to visualize MnSOD expression in p16 mutation carrier eccrine glands and nevi and compared to non-carrier tissues. Red stained SOD2 was seen in the mutation carrier tissue but non in the non-carrier tissue. The high degree of preferential staining showed that expression of this gene has utility for testing skin biopsises and is particularly desirable. In another embodiment assay of another up-regulated gene product such as GPX4, PTGDS/PGDS2, ACADM, ACS3/FACL3, KRT19, CDC25SA, BRD2, JAK1, and/or MSH5 is used in combination to improve the predictive value of a test such as a histological test. In yet another embodiment a down regulated gene (that is expressed less in a premalignant transformation tissue), such as CYP21A2, AKRIC1, BCL10, and/or FOXC1/FKHL7 is simultaneously assayed, by itself or in combination with that of another gene. In yet another embodiment, any combination of 2, 3, 4, 5, 6, 7, 8, 9, 10, 11, 12, 13, or 14 of the gene products are assayed simultaneously or sequentially from the same biopsy and the increases and/or decreases in expressions correlated with the diagnostic changes evinced in Table 5, as prognosis indicators.

In another experiment, a commercially available assay for SOD function was used to assay SOD activity in fibroblasts derived from a p16 mutation carrier and from normal non-carrier skin. To show that oxidative stress pathways activate early in the transformation process, fibroblast cultures were challenged with H2O2 and SOD activity measured. In this experiment, cultured fibroblasts were exposed (or not exposed) to 100 micromolar hydrogen peroxide for 60 minutes. Exposed cells were washed in phosphate buffered saline, counted, and sent to ZeptoMetrix Corp. (Buffalo, N.Y.) for activity measurements. Activity measurements were adjusted for cell number. The data obtained showed that V126D p16 mutation carrier samples exhibited the highest SOD activities, −34 5′UTR p16 mutation carrier samples exhibited second highest activities and that both activities decreased with hydrogen peroxide administration. Non-carrier control samples exhibited much less SOD activity.

In yet another experiment, increase in each gene in the transformation process with respect to normal control tissue was examined by room temperature PCR. In this study, gene expression of SOD2, GPX4, PTGDS/PGDS2, CDC25A, BRD2, and MSH5 were significantly increased (Table 5)

TABLE 1
Target genes (gene panel) whose expression was detectably altered during
the development of melanoma.

| | | | Vendor's | Vendor's | Mutation | | Nevus |
|---|---|---|---|---|---|---|---|
| Hs ID | Symbol | Gene Name | Clone ID | GenBank ID | Effect | p-value | Effect |
| Hs.89525 | HDGF | hepatoma-derived growth factor | 295004 | | 3.3278 | 0.0067 | −.0657 |
| | | (high-mobility group protein 1-like) | | | | | |
| Hs.96398 | GPX4 | glutathione peroxidase 4 | 298625 | | 2.9456 | 0.0262 | .4317 |
| | | (phospholipid hydroperoxidase);, | | | | | |
| | | based on sequencing; vendor's | | | | | |
| | | clone was “Human 8- | | | | | |
| | | hydroxyguanine glycosylase | | | | | |
| | | (hMMH) mRNA, complete cds”) | | | | | |
| Hs.2780 | JUND | jun D proto-oncogene | 767784 | AA418670 | 2.7501 | 0.0002 | .4477 |
| Hs.184771 | NFIC | nuclear factor I/C (CCAAT-binding | 265874 | N20996 | 2.7003 | | .8245 |
| | | transcription factor) | | | | | |
| Hs.1686 | GNA11 | guanine nucleotide binding protein | 221826 | H92232 | 2.6832 | 0.0022 | .4216 |
| | | (G protein), alpha 11 (Gq class) | | | | | |
| Hs.30954 | PMVK | phosphomevalonate kinase | 46897 | H09914 | 2.6083 | 0.0215 | −.4299 |
| Hs.75243 | BRD2 | bromodomain containing 2 | 214133 | H72520 | 2.3057 | 0.0223 | .4045 |
| | | (RING3, female sterile homeotic- | | | | | |
| | | related gene 1 (mouse homolog))) | | | | | |
| Hs.91299 | GNB2 | guanine nucleotide binding protein | 292213 | N68166 | 2.1881 | 0.0009 | .5974 |
| | | (G protein), beta polypeptide | | | | | |
| | | 2 (postmeiotic segregation | | | | | |
| | | increased 2-like 12) | | | | | |
| Hs.1435 | GMPR | guanosine monophosphate | 753775 | AA406242 | 2.1398 | 0.0469 | .0788 |
| | | reductase | | | | | |
| Hs.146354 | PRDX2 | peroxiredoxin 2 (based on | 212165 | H68845 | 2.0732 | 0.0471 | .1200 |
| | | sequencing; vendor's clone was | | | | | |
| | | “thioredoxin-dependent peroxide | | | | | |
| | | reductase 1 (thiol-specific | | | | | |
| | | antioxidant 1, natural killer- | | | | | |
| | | enhancing factor B)”) | | | | | |
| Hs.170157 | MYO5A | myosin VA (heavy polypeptide 12, | 365755 | AA025850 | 2.0464 | 0 | −.0646 |
| | | myoxin) | | | | | |
| Hs.75082 | ARHG | ras homolog gene family, member | 158086 | | 1.9407 | 0 | −.0378 |
| | | G (rho G) | | | | | |
| Hs.2706 | PLCG2 | phospholipase C, gamma 2 (based | 809981 | AA455197 | 1.9018 | 0.035 | .2172 |
| | | on sequencing; vendor's clone was | | | | | |
| | | “glutathione peroxidase 4 | | | | | |
| | | (phospholipid hydroperoxidase)”) | | | | | |
| Hs.88474 | PTGS1 | prostaglandin-endoperoxide | 325939 | | −1.7814 | 0 | −.3725 |
| | | synthase 1 (prostaglandin G/H | | | | | |
| | | synthase and cyclooxygenase) | | | | | |
| Hs.78580 | FBN1 | fibrillin 1 (based on sequencing; | 486535 | | 1.6644 | 0.05 | −.4910 |
| | | vendor's clone was “DEAD/H (Asp- | | | | | |
| | | Glu-Ala-Asp/His) box polypeptide | | | | | |
| | | 1”) | | | | | |

TABLE 2
List of genes with high differential mutation effect.

| | | | | | | |
|---|---|---|---|---|---|---|
| | | | | Mutation | Nevus | |
| Hs ID | LocusID | Symbol | Gene Name | Effect | Effect | Chromosome |

(Positive effect)

| | | | | | | |
|---|---|---|---|---|---|---|
| Hs.380718 | — | SERF2 | small EDRK-rich factor, (Gastric | 3.4812 | −.0042 | 18 |
| | | | cancer-related protein VRG107), | | | |
| | | | a.k.a. FAM2C, 4F5REL | | | |
| Hs.118838 | — | H3F3A | H3 histone, family 3A; HISTONE | 3.3658 | .0671 | |
| | | | H3.3 | | | |
| Hs.89525 | 3068 | HDGF | hepatoma-derived growth factor | 3.3278 | −.0657 | X |
| | | | (high-mobility group protein 1- | | | |
| | | | like) | | | |
| Hs.114929 | — | HLA-DRB5 | Major histocompatibility complex, | 3.0586 | −.5008 | |
| | | | class II, DR beta 5 (or HLA- | | | |
| | | | DRB1 major histocompatibility | | | |
| | | | complex, class II, DR beta 1 | | | |
| | | | or 3 | | | |
| Hs.154417 | — | KCNAB2 | potassium voltage-gated | 3.0168 | .2901 | |
| | | | channel, shaker-related | | | |
| | | | subfamily, beta member 2 | | | |
| Hs.96398 | 4968 | GPX4 | glutathione peroxidase 4 | 2.9456 | .4317 | 3 |
| | | | (phospholipid hydroperoxidase);, | | | |
| | | | based on sequencing; vendor's | | | |
| | | | clone was “Human 8- | | | |
| | | | hydroxyguanine glycosylase | | | |
| | | | (hMMH) mRNA, complete cds”) | | | |
| Hs.79706 | 5339 | PLEC1 | plectin 1, intermediate filament | 2.7728 | .3301 | 8 |
| | | | binding protein, 500 kD | | | |
| Hs.2780 | 3727 | JUND | jun D proto-oncogene | 2.7501 | .4477 | 19 |
| Hs.151031 | — | API5 | Apoptosis inhibitor 5 (N1 | 2.7245 | −.0711 | |
| | | | fibroblast growth factor 2- | | | |
| | | | interacting factor, FIF) | | | |
| Hs.184771 | 4782 | NFIC | nuclear factor I/C (CCAAT- | 2.7003 | .8245 | 19 |
| | | | binding transcription factor) | | | |
| Hs.1686 | 2767 | GNA11 | guanine nucleotide binding | 2.6832 | .4216 | 19 |
| | | | protein (G protein), alpha 11 (Gq | | | |
| | | | class) | | | |
| Hs.21278 | — | | Hs.387901, Clone 28191 | 2.6791 | −.1626 | |
| Hs.77269 | 2771 | GNAI2 | guanine nucleotide binding | 2.6439 | −.2578 | 3 |
| | | | protein (G protein); alpha | | | |
| | | | inhibiting activity polypeptide 2 | | | |
| Hs.30954 | 10654 | PMVK | phosphomevalonate kinase | 2.6083 | −.4299 | 1 |
| Hs.78979 | 2734 | GLG1 | Golgi apparatus protein 1; Golgi | 2.5118 | .2877 | 16 |
| | | | membrane sialoglycoprotein | | | |
| | | | MG160 (GLG1) | | | |
| Hs.2780 | 3727 | JUND | jun D proto-oncogene | 2.4643 | .4100 | 19 |
| Hs.21921 | — | | LOC283760 | 2.3911 | .0311 | |
| Hs.124239 | — | GCH1 | GTP cyclohydrolase 1 (dopa- | 2.3828 | −.7558 | |
| | | | responsive dystonia) | | | |
| Hs.125078 | — | | ornithine decarboxylase | 2.3353 | −.1048 | 19 |
| | | | antizyme, ORF 1 and ORF 2 | | | |
| Hs.75243 | 6046 | BRD2 | bromodomain containing 2 | 2.3057 | .4045 | 6 |
| Hs.76307 | 4681 | NBL1 | neuroblastoma, suppression of | 2.2916 | −.3661 | 1 |
| | | | tumorigenicity 1 | | | |
| Hs.17987 | 79140 | MGC1203 | hypothetical protein MGC1203 | 2.2300 | .2852 | 1 |
| Hs.91299 | 2783 | GNB2 | guanine nucleotide binding | 2.1881 | .5974 | 7 |
| | | | protein (G protein), beta | | | |
| | | | polypeptide 2 | | | |
| Hs.169055 | — | GOLGA2 | golgi autoantigen, golgin | 2.1633 | .5522 | |
| | | | subfamily a, 2; clone also assoc | | | |
| | | | w TEM6 (7p12.3) Tumor | | | |
| | | | endothelial marker 6 | | | |
| Hs.1435 | 2766 | GMPR | guanosine monophosphate | 2.1398 | .0788 | 6 |
| | | | reductase | | | |
| Hs.28728 | — | C6orf37 | chromosome 6 open reading | 2.1303 | −.6136 | |
| | | | frame 37 | | | |
| Hs.75725 | 8407 | TAGLN2 | transgelin 2 | 2.1239 | −.2963 | 6 |
| Hs.49762 | — | YWHAE | tyrosine 3- | 2.1237 | −.5338 | |
| | | | monooxygenase/tryptophan 5- | | | |
| | | | monooxygenase activation | | | |
| | | | protein, epsilon polypeptide | | | |
| Hs.2340 | 3728 | PLAK2 | junction plakoglobin | 2.0747 | −.1563 | 17 |
| | | JUP | | | | |
| Hs.146354 | 7001 | PRDX2 | peroxiredoxin 2 | 2.0732 | .1200 | 13 |
| Hs.170157 | 4644 | MYO5A | myosin VA (heavy polypeptide | 2.0464 | −.0646 | 15 |
| | | | 12, myoxin) | | | |
| Hs.73151 | — | CDC27 | Cell division cycle 27 | 2.0381 | .1294 | |
| Hs.169902 | 6513 | SLC2A1 | solute carrier family 2 (facilitated | 2.0090 | −.3959 | 1 |
| | | | glucose transporter), member 1 | | | |
| Hs.8249 | — | SH3BGRL3 | Socius; SH3 domain binding | 2.0036 | .1486 | |
| | | SOC | glutamic acid-rich protein like 3; | | | |
| | | | TNF inhibitory protein; | | | |
| | | | SH3BGRL3-like protein | | | |
| Hs.27742 | — | | KIAA1026 protein | 1.9936 | .3127 | |
| Hs.28914 | 353 | APRT | adenine | 1.9428 | .3672 | 16 |
| | | | phosphoribosyltransferase | | | |
| Hs.75082 | 391 | ARHG | ras homolog gene family, | 1.9407 | −.0378 | 11 |
| | | | member G (rho G) | | | |
| — | — | GAPD | glyceraldehyde-3-phosphate | 1.9336 | .1604 | |
| | | | dehydrogenase | | | |
| Hs.3321 | — | IRX3 | Iroquois-class homeodomain | 1.9138 | .2018 | |
| | | | protein | | | |
| Hs.77196 | 6709 | SPTAN1 | spectrin, alpha, non-erythrocytic | 1.9037 | .0730 | 9 |
| | | | 1 (alpha-fodrin) | | | |
| Hs.2706 | 2879 | PLCG2 | phospholipase C, gamma 2 | 1.9018 | .2172 | 19 |
| | | | (based on sequencing; vendor's | | | |
| | | | clone was “glutathione | | | |
| | | | peroxidase 4 (phospholipid | | | |
| | | | hydroperoxidase)”) | | | |
| Hs.72082 | — | SDC4 | syndecan 4 (amphiglycan, | 1.8986 | .1135 | |
| | | | ryudocan) | | | |
| Hs.56205 | 3638 | INSIG1 | insulin induced gene 1 | 1.8862 | −.2271 | 7 |
| Hs.255876 | — | RAP2A | RAP2A, member of RAS | 1.8762 | .7061 | |
| | | | oncogene family (rap2 mRNA for | | | |
| | | | ras-related protein) | | | |
| Hs.255408 | — | DGAT2 | Diacylglycerol O-acyltransferase | 1.8748 | −.1401 | |
| | | | homolog 2 (mouse) | | | |
| Hs.74456 | — | | hypothetical protein FLJ10891 | 1.8471 | .0979 | |
| Hs.78880 | 10994 | ILVBL | ilvB (bacterial acetolactate | 1.8351 | .0516 | 19 |
| | | | synthase)-like | | | |
| — | — | IGKC | myosin-reactive immunoglobulin | 1.8290 | .2903 | |
| | | | light chain variable region | | | |
| | | | mRNA; Anti-streptococcal/anti- | | | |
| | | | myosin immunoglobulin kappa | | | |
| | | | light chain variable region | | | |
| Hs.75268 | 6484 | SIAT4C | sialyltransferase 4C (beta- | 1.8229 | −.0865 | 11 |
| | | | galactosidase alpha-2,3- | | | |
| | | | sialytransferase) | | | |
| Hs.28914 | 353 | APRT | adenine | 1.8198 | .2570 | 16 |
| | | | phosphoribosyltransferase | | | |
| Hs.75564 | 977 | COX7A2L | cytochrome c oxidase (COX) | 1.8132 | .3575 | 11 |
| | | COX7RP | subunit VIIa polypeptide 2-like | | | |
| | | | (COX7A2L) (based on sequence | | | |
| | | | confirmation; vendor clone was | | | |
| | | | CD151 antigen) | | | |
| Hs.78534 | — | | | 1.8002 | .2205 | |
| Hs.79149 | — | | | 1.7967 | .1877 | |
| Hs.66369 | — | | | 1.7964 | .1696 | |
| Hs.73151 | — | | | 1.7900 | −.0217 | |
| Hs.23037 | — | | | 1.7818 | −.0730 | |
| Hs.203656 | — | | | 1.7352 | −.3218 | |
| Hs.99923 | 3963 | LGALS7 | lectin, galactoside-binding, | 1.7227 | .4392 | 19 |
| | | | soluble, 7 (galectin 7) | | | |
| Hs.75184 | 1116 | CHI3L1 | chitinase 3-like 1 (cartilage | 1.7224 | .1916 | 1 |
| | | | glycoprotein-39) | | | |
| Hs.699 | 5479 | PPIB | peptidylprolyl isomerase B | 1.7071 | −.2852 | 15 |
| | | | (cyclophilin B) | | | |
| Hs.183994 | 5499 | PPP1CA | protein phosphatase 1, catalytic | 1.6954 | .2353 | 11 |
| | | | subunit, alpha isoform | | | |
| Hs.1139 | — | | | 1.6816 | −.3148 | |
| Hs.215595 | 2782 | GNB1 | guanine nucleotide binding | 1.6781 | .1134 | 1 |
| | | | protein (G protein), beta | | | |
| | | | polypeptide 1 | | | |
| Hs.78580 | 1653 | DDX1 | DEAD/H (Asp-Glu-Ala-Asp/His) | 1.6644 | −.4910 | 2 |
| | | | box polypeptide 1 | | | |
| Hs.6793 | 5050 | PAFAH1B3 | platelet-activating factor | 1.6576 | .5496 | 19 |
| | | | acetylhydrolase, isoform lb, | | | |
| | | | gamma subunit (29 kD) | | | |
| Hs.83753 | 6628 | SNRPB | small nuclear ribonucleoprotein | 1.6513 | .0107 | 20 |
| | | | polypeptides B and B1 | | | |
| Hs.155109 | 3294 | HSD17B2 | hydroxysteroid (17-beta) | 1.6443 | −.1607 | 16 |
| | | | dehydrogenase 2 | | | |
| Hs.172609 | 4924 | NUCB1 | nucleobindin 1 | 1.6420 | .1071 | 19 |
| Hs.621 | 3958 | LGALS3 | lectin, galactoside-binding, | 1.6289 | .1847 | 14 |
| | | | soluble, 3 (galectin 3) | | | |
| Hs.211584 | 4747 | NEFL | neurofilament, light polypeptide | 1.6203 | .2006 | 8 |
| | | | (68 kD) | | | |
| Hs.91096 | 11074 | TRIM31 | tripartite motif-containing 31 | 1.6117 | −1.2755 | 6 |
| Hs.8272 | 5730 | PTGDS | prostaglandin D2 synthase | 1.6092 | .3256 | 9 |
| | | | (21 kD, brain) | | | |
| Hs.95998 | 2395 | FRDA | Friedreich ataxia | 1.6054 | −1.2797 | 9 |
| Hs.75108 | 6050 | RNH | ribonuclease/angiogenin | 1.5902 | .0051 | 11 |
| | | | inhibitor | | | |
| Hs.86358 | — | | | 1.5902 | −.0322 | |
| Hs.79000 | 2596 | GAP43 | growth associated protein 43 | 1.5839 | −1.2862 | 3 |
| Hs.75465 | — | | | 1.5792 | −1.1526 | |
| Hs.153910 | — | | | 1.5684 | .3934 | |
| — | — | | | 1.5625 | .4392 | |
| Hs.243960 | 57447 | NDRG2 | N-myc downstream-regulated | 1.5504 | .2880 | 14 |
| | | | gene 2 | | | |
| Hs.74122 | 837 | CASP4 | caspase 4, apoptosis-related | 1.5427 | −.2444 | 11 |
| | | | cysteine protease | | | |
| Hs.73931 | 3119 | HLA-DQB1 | major histocompatibility | 1.5414 | −.1692 | 6 |
| | | | complex, class II, DQ beta 1 | | | |
| Hs.79136 | 25800 | LIV-1 | LIV-1 protein, estrogen | 1.5357 | −.0844 | 18 |
| | | | regulated | | | |
| Hs.6793 | 5050 | PAFAH1B3 | platelet-activating factor | 1.5202 | .6490 | 19 |
| | | | acetylhydrolase, isoform lb, | | | |
| | | | gamma subunit (29 kD) | | | |
| Hs.58297 | 83852 | CLLD8 | CLLL8 protein | 1.5117 | −.2642 | 13 |
| Hs.74497 | 4904 | NSEP1 | nuclease sensitive element | 1.5084 | .0853 | 1 |
| | | | binding protein 1 | | | |

(Negative effect)

| | | | | | | |
|---|---|---|---|---|---|---|
| — | — | C17orf26 | chromosome 17 open | −2.8941 | .1929 | |
| | | | reading frame 26 | | | |
| Hs.24605 | — | MBNL1 | muscleblind-like | −2.7495 | −.2240 | 3 |
| | | | (Drosophila) | | | |
| Hs.53875 | — | HLA-DQA1 | MHC class II DQ alpha, HLA- | −2.5737 | −.1567 | |
| | | | DQA2, HLA class II | | | |
| | | | histocompatibility antigen, | | | |
| | | | DQ | | | |
| Hs.34578 | 10402 | ST3GALVI | alpha2,3-sialyltransferase | −2.5276 | 2.2189 | 3 |
| Hs.155376 | 3043 | HBB | hemoglobin, beta | −2.5074 | 2.3903 | 11 |
| Hs.53875 | — | HLA-DQA1 | MHC class II DQ alpha, | −2.4148 | −.0150 | |
| | | | HLA-DQA2, HLA class II | | | |
| | | | histocompatibility antigen, | | | |
| | | | DQ | | | |
| Hs.159867 | — | HNRPH1 | Heterogeneous nuclear | −2.2619 | 1.9925 | |
| | | | ribonucleoprotein H1 (H) | | | |
| Hs.9950 | 23480 | SEC61G | Sec61 gamma | −2.2469 | −.4419 | 7 |
| Hs.46452 | 4250 | SCGB2A2 | secretoglobin, family 2A, | −2.2138 | .2577 | 11 |
| | | | member 2 (Human | | | |
| | | | mammaglobin) | | | |
| Hs.198253 | 3117 | HLA-DQA1 | major histocompatibility | −2.0359 | .2124 | 6 |
| | | | complex, class II, DQ alpha 1 | | | |
| Hs.198253 | 3117 | HLA-DQA1 | major histocompatibility | −1.7909 | .0329 | 6 |
| | | | complex, class II, DQ alpha 1 | | | |
| Hs.88474 | 5742 | FBN1 | fibrillin 1 (based on | −1.7814 | −.3725 | 9 |
| | | | sequencing; vendor's clone | | | |
| | | | was “DEAD/H (Asp-Glu-Ala- | | | |
| | | | Asp/His) box polypeptide 1”) | | | |
| Hs.1066 | — | SNRPE | Small nuclear | −1.7664 | −.8624 | |
| | | | ribonucleoprotein polypeptide E | | | |
| Hs.78225 | 301 | ANXA1 | annexin A1 | −1.7603 | −.7939 | 9 |
| Hs.108485 | — | MYL4 | atrial/embryonic alkali | −1.6968 | −.7956 | |
| | | (MLC1) | myosin light chain; Myosin | | | |
| | | | light chain 1, embryonic | | | |
| | | | muscle/atrial isoform | | | |
| Hs.82547 | 5918 | RARRES1 | retinoic acid receptor | −1.6593 | −.9566 | 3 |
| | | | responder (tazarotene | | | |
| | | | induced) 1 | | | |
| Hs.104318 | — | TRADD | TNFRSF1A-associated via | −1.5861 | .5000 | |
| | | | death domain | | | |
| Hs.184014 | 6160 | RPL31 | ribosomal protein L31 | −1.5574 | −.7484 | 2 |
| Hs.118838 | — | | Transcribed sequence with | −1.5426 | −1.5446 | |
| | | | moderate similarity to LZ16 | | | |
| | | | protein ref: NP_037407.1 | | | |
| | | | (H. sapiens) | | | |
| Hs.82758 | — | COX6C | CYTOCHROME C OXIDASE | −1.5313 | −.7253 | |
| | | | POLYPEPTIDE VIC | | | |
| | | | PRECURSOR | | | |
| Hs.84898 | — | NKTR | natural killer-tumor | −1.5033 | −.7569 | |
| | | | recognition sequence | | | |
| Hs.166361 | — | | Homo sapiens mRNA; cDNA | −1.5019 | −.1763 | |
| | | | DKFZp564F112 (from clone | | | |
| | | | DKFZp564F112) | | | |

TABLE 3
Annotation of genes with high differential effects.
BIOLOGICAL_PROCESS

| | Selected | Total | |
|---|---|---|---|
| Gene Ontology: biological_process | Genes | Genes | Score* |
| cell communication | 14 | 713 | |
| cell adhesion | 3 | 99 | |
| response to external stimulus | 4 | 274 | |
| response to biotic stimulus | 3 | 205 | |
| signal transduction | 7 | 446 | |
| cell surface receptor linked signal | 4 | 145 | |
| transduction | | | |
| G-protein coupled receptor protein | 4 | 60 | 4.34 |
| signaling pathway | | | |
| intracellular signaling cascade | 3 | 84 | |
| small GTPase mediated signal transduction | 2 | 23 | 5.66 |
| cell growth and/or maintenance | 23 | 1296 | |
| cell organization and biogenesis | 2 | 56 | |
| cytoplasm organization and biogenesis | 2 | 34 | 3.83 |
| cell proliferation | 3 | 155 | |
| Metabolism | 16 | 872 | |
| Biosynthesis | 2 | 104 | |
| Catabolism | 2 | 84 | |
| macromolecule catabolism | 2 | 77 | |
| nucleobase, nucleoside, nucleotide and | 7 | 336 | |
| nucleic acid metabolism | | | |
| RNA metabolism | 2 | 59 | |
| RNA processing | 2 | 58 | |
| mRNA processing | 2 | 39 | |
| mRNA splicing | 2 | 30 | 4.34 |
| Transcription | 3 | 202 | |
| transcription, DNA-dependent | 3 | 194 | |
| transcription, from Pol II promoter | 2 | 73 | |
| phosphate metabolism | 2 | 93 | |
| Transport | 3 | 135 | |
| vesicle-mediated transport | 2 | 30 | 4.34 |
| developmental processes | 4 | 309 | |
| embryogenesis and morphogenesis | 3 | 230 | |
| histogenesis and organogenesis | 2 | 196 | |
| ectoderm development | 2 | 97 | |
| Neurogenesis | 2 | 77 | |
| Reproduction | 2 | 19 | 6.85 |
| Gametogenesis | 2 | 16 | 8.14 |
| Spermatogenesis | 2 | 13 | 10.02 |
| Obsolete | 3 | 188 | |
| Oncogenesis | 2 | 107 | |
| physiological processes | 2 | 119 | |
| Total Genes | 79 | 5144 | |

MOLECULAR_FUNCTION

| | Selected | Total | |
|---|---|---|---|
| Gene Ontology: molecular_function | Genes | Genes | Score* |
| Enzyme | 19 | 689 | 1.80 |
| Hydrolase | 10 | 276 | 2.36 |
| hydrolase, acting on acid anhydrides | 7 | 92 | 4.95 |
| hydrolase, acting on acid anhydrides, in | 7 | 88 | 5.18 |
| phosphorus-containing anhydrides | | | |
| ATPase | 2 | 46 | |
| GTPase | 5 | 37 | 8.80 |
| heterotrimeric G-protein GTPase | 4 | 8 | 32.56 |
| heterotrimeric G-protein GTPase, | 2 | 4 | 32.56 |
| alpha-subunit | | | |
| heterotrimeric G-protein GTPase, | 2 | 2 | 65.11 |
| beta-subunit | | | |
| hydrolase, acting on ester bonds | 2 | 83 | |
| Kinase | 3 | 149 | |
| Oxidoreductase | 3 | 66 | 2.96 |
| oxidoreductase, acting on peroxide | 2 | 7 | 18.60 |
| as acceptor | | | |
| Peroxidase | 2 | 6 | 21.70 |
| Transferase | 2 | 107 | |
| transferase, transferring glycosyl groups | 2 | 26 | 5.01 |
| ligand binding or carrier | 10 | 719 | |
| carbohydrate binding | 2 | 6 | 21.70 |
| sugar binding | 2 | 6 | 21.70 |
| Lectin | 2 | 4 | 32.56 |
| nucleic acid binding | 5 | 385 | |
| DNA binding | 5 | 281 | |
| transcription factor | 3 | 189 | |
| protein binding | 2 | 240 | |
| cytoskeletal protein binding | 2 | 37 | |
| structural molecule | 3 | 83 | |
| structural constituent of cytoskeleton | 3 | 21 | 9.30 |
| Transporter | 4 | 146 | |
| electron transporter | 3 | 39 | 5.01 |
| Total Genes | 79 | 5144 | |

CELLULAR_COMPONENT

| | Selected | Total | |
|---|---|---|---|
| Gene Ontology: cellular_component | Genes | Genes | Score* |
| Cell | 24 | 1239 | |
| cell fraction | 5 | 188 | |
| membrane fraction | 4 | 127 | |
| soluble fraction | 2 | 66 | |
| Intracellular | 22 | 1117 | |
| Cytoplasm | 15 | 524 | 1.86 |
| cytoskeleton | 3 | 88 | |
| intermediate filament cytoskeleton | 2 | 12 | 10.85 |
| intermediate filament | 2 | 12 | 10.85 |
| endoplasmic reticulum | 2 | 50 | |
| mitochondrion | 2 | 81 | |
| Nucleus | 3 | 340 | |
| plasma membrane | 4 | 344 | |
| Membrane | 3 | 126 | |
| Extracellular | 5 | 161 | |
| extracellular space | 4 | 93 | 2.80 |
| Total Genes | 79 | 5144 | |

BIOLOGICAL_PROCESS

| | Selected | | |
|---|---|---|---|
| Gene Ontology: biological_process | Genes | Total Genes | Score* |
| cell growth and/or maintenance | 5 | 1296 | |
| metabolism | 4 | 872 | |
| lipid metabolism | 3 | 74 | 11.10 |
| physiological processes | 2 | 119 | 4.60 |
| Total Genes | 19 | 5204 | |

MOLECULAR_FUNCTION

| | Selected | | |
|---|---|---|---|
| Gene Ontology: molecular_function | Genes | Total Genes | Score* |
| Enzyme | 2 | 689 | |
| ligand binding or carrier | 2 | 719 | |
| Total Genes | 19 | 5204 | |

CELLULAR_COMPONENT

| | Selected | | |
|---|---|---|---|
| Gene Ontology: cellular_component | Genes | Total Genes | Score* |
| Cell | 4 | 1239 | |
| Intracellular | 2 | 1117 | |
| Cytoplasm | 2 | 524 | |
| Membrane | 2 | 126 | 4.35 |
| Integral membrane protein | 2 | 77 | 7.11 |
| Total Genes | 19 | 5204 | |

*Scores significant at the 95.0% level are calculated according to a chi-square test
M-effect < −1.5

TABLE 4
Functional classification of statistically significant gene clusters.

| | | | | | | |
|---|---|---|---|---|---|---|
| | | | | Mutation | P-value | Nevus |
| Hs ID | LocusID | Symbol | Gene Name | Effect | M-Effect | Effect |

Cell adhesion

| | | | | | | |
|---|---|---|---|---|---|---|
| Hs.2340 | 3728 | JUP | junction plakoglobin | 2.0747 | 0.0843 | −.1563 |
| Hs.75564 | 977 | CD151 | CD151 antigen | 1.8132 | 0.049 | .3575 |

GPCR signaling

| | | | | | | |
|---|---|---|---|---|---|---|
| Hs.77269 | 2771 | GNAI2 | guanine nucleotide binding | 2.6439 | 0.0923 | −.2578 |
| | | | protein (G protein), alpha | | | |
| | | | inhibiting activity polypeptide 2 | | | |
| Hs.91299 | 2783 | GNB2 | guanine nucleotide binding | 2.1881 | 0.0009 | .5974 |
| | | | protein (G protein), beta | | | |
| | | | polypeptide 2 | | | |

Oxido-reductases

| | | | | | | |
|---|---|---|---|---|---|---|
| Hs.1435 | 2766 | GMPR | guanosine monophosphate | 2.1398 | 0.0469 | .0788 |
| | | | reductase | | | |
| Hs.146354 | 7001 | PRDX2 | peroxiredoxin 2 | 2.0732 | 0.0471 | .1200 |
| Hs.2706 | 2879 | GPX4 | glutathione peroxidase 4 | 1.9018 | 0.035 | .2172 |
| | | | (phospholipid hydroperoxidase) | | | |

G-protein GTPases

| | | | | | | |
|---|---|---|---|---|---|---|
| Hs.1686 | 2767 | GNA11 | guanine nucleotide binding | 2.6832 | 0.0022 | .4216 |
| | | | protein (G protein), alpha 11 (Gq | | | |
| | | | class) | | | |
| Hs.77269 | 2771 | GNAI2 | guanine nucleotide binding | 2.6439 | 0.0923 | −.2578 |
| | | | protein (G protein), alpha | | | |
| | | | inhibiting activity polypeptide 2 | | | |
| Hs.91299 | 2783 | GNB2 | guanine nucleotide binding | 2.1881 | 0.0009 | .5974 |
| | | | protein (G protein), beta | | | |
| | | | polypeptide 2 | | | |

Extracellular proteins

| | | | | | | | |
|---|---|---|---|---|---|---|---|
| Hs.89525 | 3068 | HDGF | hepatoma-derived growth factor | 3.3278 | 0.0067 | −.0657 | −.6760 |
| | | | (high-mobility group protein 1- | | | | |
| | | | like) | | | | |
| Hs.172609 | 4924 | NUCB1 | nucleobindin 1 | 1.6420 | 0.0192 | .1071 | .6152 |

Membrane proteins

| | | | | | | | |
|---|---|---|---|---|---|---|---|
| Hs.75564 | 977 | CD151 | CD151 antigen | 1.8132 | 0.049 | .3575 | .6728 |
| Hs.8272 | 5730 | PTGDS | prostaglandin D2 synthase | 1.6092 | 0.0875 | .3256 | .0724 |
| | | | (21 kD, brain) | | | | |
| Hs.24447 | 10280 | SR-BP1 | sigma receptor (SR31747 | 1.4677 | 0 | .0895 | .0712 |
| | | | binding protein 1) | | | | |

Regulation of cell proliferation (relaxed expression > 1.3)

| | | | | | | | |
|---|---|---|---|---|---|---|---|
| Hs.75082 | 391 | ARHG | ras homolog gene family, | 1.9407 | 0 | −.0378 | −.0370 |
| | | | member G (rho G) | | | | |
| Hs.239737 | 1487 | CTBP1 | C-terminal binding protein 1 | 1.4511 | 0 | −.4425 | −.5658 |

Kinase (relaxed expression > 1.3)

| | | | | | P-value | | |
|---|---|---|---|---|---|---|---|
| Hs ID | LocusID | Symbol | Gene Name | M Effect | M-Effect | N Effect | M × N Inter |
| Hs.30954 | 10654 | PMVK | phosphomevalonate kinase | 2.6083 | 0.0215 | −.4299 | .0972 |
| Hs.75243 | 6046 | BRD2 | bromodomain containing 2 | 2.3057 | 0.0223 | .4045 | .5410 |
| Hs.6241 | 5295 | PIK3R1 | phosphoinositide-3-kinase, | 1.3960 | 0.0778 | −.2625 | −.2550 |
| | | | regulatory subunit, polypeptide 1 | | | | |
| | | | (p85 alpha) | | | | |
| Hs.6241 | 5295 | PIK3R1 | phosphoinositide-3-kinase, | 1.3872 | 0.0001 | −.4855 | −.6028 |
| | | | regulatory subunit, polypeptide 1 | | | | |
| | | | (p85 alpha) | | | | |

TABLE 5
Particularly Useful Target Genes

| | | | Microarray | |
|---|---|---|---|---|
| | | | Fold Change | |
| | | | (Carrier:Non- | ¹RT-PCR |
| Gene Name | Pathway | Function | carrier) | Fold Change |
| Superoxide dismutase 2 (SOD2) | Oxidative | Dismutation of superoxide anions | ↑ 2.8 | ²Increased by |
| | stress | | | IHC and |
| | | | | activity |
| ³Glutathione peroxidase 4 (GPX4) | Oxidative | Reduces phospholipid hydroperoxides | ↑ 7.9 | ↑ 2.3 |
| | stress | | | |
| Prostaglandin D2 synthase | Oxidative | Enzyme producing prostaglandin D2 | ↑ 3.0 | ↑ 2.8 |
| (PTGDS/PGDS2) | stress | (anti-inflammatory) | | |
| Cytochrome P450 family 21, AKA | Oxidative | P450 superfamily involved in redox | ↓ 4.8 | |
| steroid 21 hydroxylase (CYP21A2) | stress | partner interaction | | |
| Acyl-Coenzyme A dehydrogenase | Oxidative | Fatty acid beta oxidation | ↑ 2.4 | |
| (ACADM) | stress | | | |
| Aldo-keto reductase family 1, member | Oxidative | Keto-steroid reductase and | ↓ 3.3 | |
| C1 (AKR1C1) | stress | hydroxysteroid oxidase activities | | |
| Fatty acid coenzyme A ligase | Oxidative | Brain predominant fatty acid | ↑ 2.4 | |
| (ACS3/FACL3) | stress | metabolism | | |
| Keratin 19 (KRT19) | Possible | Poorly characterized keratin, but | ↑ 8.2 | |
| | oxidative | similar to keratins induced by | | |
| | stress | oxidative stress (e.g. keratin 6a) | | |
| Cell division cycle 25A (CDC25A) | Cell cycle | Promotes CDC2-mediated cell | ↑ 2.5 | ↑ 2.0 |
| | regulation | division | | |
| Bromodomain-containing 2 protein | Cell cycle | A RING3 type of mitogen activated | ↑ 5.0 | ↑ 4.7 |
| (BRD2) | regulation | kinase | | |
| B-cell CLL/lymphoma 10 (BCL10) | Cell cycle | Oncogene, activates NFKB and | ↓ 2.5 | |
| | regulation | induces JNK | | |
| Janus kinase 1 (JAK1) | Cell cycle | Transduces signals from IFN, IL-3 & | ↑ 7.3 | |
| | regulation | IL-6 via STATS | | |
| Forkhead box C1 (FOXC1/FKHL7) | Cell cycle | TGF-beta induced tumor suppressor | ↓ 3.2 | |
| | regulation | transcription factor | | |
| MutS homolog 5(MSH5) | DNA repair | DNA mismatch repair enzyme | ↑ 2.0 | ↑ 4.7 |

What is claimed is:

1. A method for detecting a tumor or a pre-malignant transformation in a mammal, comprising: assaying the level of expression of at least one gene or protein and/or the activity of at least one protein in a sample obtained from a mammal wherein said sample is from a region of said mammal that is suspected to be precancerous or cancerous or is a bodily fluid of said mammal, and wherein said gene or protein is selected from the genes listed in Table 1, Table 2, and/or Table 5.

2. The method according to claim 1, wherein a change in baseline level of expression of said gene or protein is predictive of a tumor or a pre-malignant transformation in said mammal.

3. The method according to claim 1, wherein the presence of a tumor or a pre-malignant transformation is indicated by altered expression of a set of genes and/or proteins in said sample.

4. The method according to claim 1, wherein said expression level or protein activity level is measured by comparison to the expression level or activity level in a control sample.

5. The method according to claim 1, wherein said assay measures altered production of mRNA transcribed from said at least one gene.

6. The method according to claim 1, wherein the assay measures altered production of the protein product of said at least one gene.

7. The method according to claim 1, wherein the assay measures altered activity of a protein in said sample.

8. The method according to claim 6, wherein said assay is carried out using a method selected from the group consisting of: genetic microarray analysis; quantitative RT-PCR; and Northern blot.

9. The method according to claim 7, wherein said assay is carried out using a method selected from the group consisting of Western blot, ELISA; immuno-histochemistry (IHC), in-situ hybridization (ISH), fluorescence-based in-situ hybridization (FISH), and a proteomics array.

10. The method according to claim 7, wherein said assay measures specific enzymatic activities of said protein.

11. The method according to claim 1, wherein said biological sample is a skin tissue.

12. A method of inhibiting tumor growth or preventing tumorigenesis in a subject, comprising administering to a patient suffering from a tumor or predisposed to a tumor a composition that alters expression of at least one gene listed in Table 1, Table 2, and/or Table 5, that alters expression of at least one protein product of a gene listed in Table 1, Table 2, and/or Table 5, and/or inhibits the activity of at least one protein product of a gene listed in Table 1, Table 2, and/or Table 5.

13. The method according to claim 11, wherein the composition affects the expression of a target gene or protein that induces melanoma.

14. The method according to claim 12, wherein the composition comprises a compound selected from the group consisting of an antisense oligonucleotide, an oligonucleotide that binds to mRNA to form a triplex, an RNAi molecule, a siRNA, an RNAi, an miRNA, a shRNA or a nucleic acid molecule encoding a siRNA, an RNA, an miRNA, or a shRNA.

15. The method according to claim 12, wherein the composition comprises a human antibody.

16. The method according to claim 1, wherein the assay is carried out using a kit, wherein the kit comprises primers or probe that specifically bind or hybridize, under stringent condition, with nucleic acid molecules identified by the genes in Table 1, Table 2, and/or Table 5.

17. The method according to claim 16, wherein the detection is carried out using a kit suitable for performing PCR, and wherein the kit comprises primers specific for the amplification of nucleic acid molecules identified by the genes in Table 1, Table 2 and/or Table 5.

18. A method for determining the efficacy of a therapeutic treatment regimen in a patient, comprising:
a) measuring expression levels of a gene or protein or activity of a protein in a first biological sample obtained from the patient, thereby generating data for a test level, wherein the gene or protein is selected from the panel consisting of the genes or their resulting proteins as listed in Table 1, Table 2, and/or Table 5;
b) administering the treatment regimen to the patient;
c) measuring the expression levels of the gene or protein or activity of a protein in a second biological sample from the patient at a time following administration of the treatment regimen; and
d) comparing the expression levels of the gene or protein or the activity levels of the protein in the first and the second biological samples, wherein data showing normalization in the levels in the second biological sample relative to the first biological sample indicates that the treatment regimen is effective in the patient.

19. A method for treating melanoma or a pre-malignant transformation in a mammalian tissue, comprising contacting the tissue with a modulating agent that interacts with at least one of the genes listed in Table 1 and/or Table 2 or its respective protein product and thereby modifies its function or activity.

20. The method according to claim 19, wherein the tissue is a skin tissue.

21. The method according to claim 20, wherein the agent is a siRNA, miRNA, an antisense RNA, an antisense DNA, a decoy molecule, or a decoy DNA.
